(19) United States
(12) Patent Application Publication
(10) Pub. No.: US 2004/0033943 A1
(43) Pub. Date: Feb. 19, 2004

(54) Hcn polypeptides and polynucleotides and their use in therapy

(76) Inventors: Paul Johannes Leonardus Maria Strijbos, Harlow (GB); Stewart Bates, Harlow (GB); Israel Simon Gloger, Harlow (GB); Ceri Hywel Davies, Harlow (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/311,795

(22) PCT Filed: Jul. 3, 2001
(86) PCT No.: PCT/GB01/02959

(30) Foreign Application Priority Data
Jul. 3, 2000 (GB) 0016360.0
Nov. 3, 2000 (GB) 0026946.4

(51) Int. Cl. A61K 48/00; A61K 38/17

(52) U.S. Cl. 514/12; 514/44

(57) ABSTRACT

The invention relates to newly identified uses of HCN channel polypeptides and polynucleotides encoding such polypeptides, to their use in therapy and in identifying compounds which may be antagonists and/or inhibitors which are potentially useful in therapy, and to production of such polypeptides and polynucleotides.

The present invention relates to newly identified uses of human hyperpolarisation-activated, cyclic nucleotide-gated (HCN) channel polypeptides and polynucleotides encoding such polypeptides, to their use in therapy and in identifying compounds, which may be agonists or antagonists, which are potentially useful in therapy. The invention further relates to newly identified HCN polypeptides and polynucleotides.

Hyperpolarisation-activated cation currents or anomalous rectifier currents, most commonly referred to as Iₕ (and alternatively referred to as I_{Q} or I_{f}) (Halliwell & Adams (1982) Brain Res 250 71-92; Mayer & Westbrook (1983) J. Physiol. 340, 1945; DiFrancesco et al (1986) J. Physiol. 377, 61-88; Spain et al (1987) J. Neurophysiol. 57, 1555-1576; McCormick & Pape (1990) J. Physiol. 431, 319-342; Maccaferri et al (1993) J. Neurophysiol. 69,2129-2136), were originally observed in heart where they have been shown to be important in the pacemaking activity underlying rhythmical heart beat. Since then it has become increasingly clear that tissues and organs other than heart also express Iₕ, including smooth muscle, endothelium and both inhibitory and excitatory neurons of the central nervous system. As with other ion channels, Iₕ displays specialized biophysical properties that are specifically suited to the physiological roles it plays throughout the body. Thus, Iₕ is inactive at depolarized membrane potentials, where action potentials are firing, but is turned on by hyperpolarization such that at membrane potentials more negative than −50 mV Iₕ is activated and passes cations into the cell causing a slow depolarization that deactivates upon continued depolarization. These particular characteristics arise from the specialized structural features of the ion channel subunits that generate the Iₕ current. In particular, the Iₕ current appears to be mediated by a new family of ion channels termed hyperpolarisation-activated cyclic nucleotide-gated (HCN) channels. These channels form a sub-family of the superfamily of voltage-gated cation channels and to date, the primary sequences of four cDNAs encoding mammalian HCN channels have been cloned, as well as a cDNA encoding an HCN channel from sea urchin sperm. Structural analysis of these channels identifies HCN channels as cousins of voltage gated K⁺ channels that display properties of cyclic nucleotide gated non-selective channels, the plant inwardly rectifying K⁺ channel KAT1 and the mammalian HERG K⁺ channels. In particular, HCN channel subunits contain six transmembrane helices (S1-S6), an ion-conducting P region between the fifth and sixth segment and a cyclic nucleotide binding domain in the C-terminus. The amino acid sequences of HCN1-4 have an overall identity of about 60% across the coding region, with up to 90% identity across the transmembrane domains and cyclic nucleotide-binding pocket. Electrophysiological analysis has revealed that HCN channels are non-selective cation channels that are permeable to both sodium and potassium ions. They possess a relatively small single channel conductance (around 1 pS in heart) and are inhibited, in a non use-dependent manner, by extracellular Cs⁺ concentrations in the 0.1-1 mM range or N-ethyl-1,6-dihydro-1,2-dimethyl-6-(methylimino)-N-phenyl-4-pyrimidinamine hydrochloride (ZD7288) in the 1-300 μM range (Bosmith et al (1993) Br. J. Pharmacol. 110, 343-; Harris & Constanti, (1995) J. Neurophysiol. 74, 2366-2378; Gasparini & DiFrancesco (1997) Pflügers Arch 435, 99-106). Binding of cyclic nucleotides to the cyclic nucleotide binding pocket most commonly results in a depolarizing shift in the steady state activation curve of Iₕ and possession of this binding pocket enables wide ranging transmitter systems to modulate the activity of this current (DiFrancesco et al (1986) supra; Bobker & Williams (1989) Neuron 2, 1530-1540; McCormick & Pape (1990) supra; DiFrancesco (1991) J. Physiol. 434, 2340; Banks et al (1993) J. Neurophysiol. 70, 1420-1432; Jiang et al (1993) J. Physiol. 450, 455468; Travagli & Gillis (1994) J. Neurophysiol. 71, 1308-1317; Ingram & Williams (1994) Neuron 13, 179-186). Thus, for example, activation of β-adrenergic receptors in the heart stimulates adenylyl cyclase activity which raises cAMP levels which, in turn, increases Iₕ activity and accelerates membrane depolarization. This, combined with other effects of β-adrenergic receptor activation, can result in a doubling in cardiac cell firing rate. Whilst the heart provides a clear illustration of one of the physiological functions fulfilled by HCN channels, activation of these channels, and modification of their activity by cellular processes that regulate cAMP production (e.g. via activation of G protein coupled receptors), in other regions of the body are likely to be equally important in determining the mental and physical well-being of an individual. In this respect, activation of HCN channels and generation of Iₕ has been shown to be critically involved in 1) determining neuronal resting membrane potentials, 2) regulating the response of neurons to hyperpolarising currents, 3) generating ‘pacemaker’ potentials that control the rate of rhythmic oscillations and 4) modulating calcium-independent neurotransmitter release; cellular processes that are critical for physiological functions such as sleep cycles, cognition and hormone secretion. Interestingly, whilst most of these physiological functions are likely to result from the electrophysiological characteristics of HCN channels it has recently been suggested that these channels may produce some of their actions by non-electrophysiological interactions with intracellular processes (e.g. microtubule and actin transport systems). As such, HCN channels exhibit highly diverse functions at the molecular, cellular and physiological levels and provide a useful target for therapeutic intervention in the treatment of human diseases relating to peripheral or CNS dysfunction. One area of interest is stroke where inhibitors of HCN function may reduce the neuronal overexcitability that initiates neurodegeneration by inhibiting glutamate release and reducing the probability of action potential firing. Conversely, activators of HCN channel function may also be neuroprotective since activation of HCN channels in inhibitory circuits may potentially counterbalance the increased activity in excitatory circuits observed during neurodegenerative insults.

The present invention is based on the finding that blocking the activation of HCN channels confers protection against neuronal cell death in organotypic hippocampal slice cultures subjected to oxygen and glucose deprivation, as well as in dispersed primary hippocampal neurons subjected to excitotoxicity. Further, the HCN channels, in particular the HCN1 and HCN4 channels, have been shown to be upregulated in a mouse model of stroke (permanent middle cerebral artery occlusion model). These two lines of evidence show that HCN channels are strong candidates for targets for therapeutic intervention for the prevention and/or treatment of diseases such as stroke, epilepsy, ischaemia, head injury, Alzheimer's disease, and also learning, memory and attention disorders. Neuroprotection is a major therapeutic target of the pharmaceutical industry and there is a clear need for the development of effective treatments for this important therapeutic goal.

Further, HCN channels are potential targets for therapeutic intervention for the treatment of pain, gut disorders, in particular Irritable Bowel Syndrome (IBS) and sleep disorders.

Thus the present invention provides for the use of a compound selected from:

(a) a HCN channel polypeptide, or a fragment thereof;

(b) a compound which inhibits an HCN channel polypeptide;

(c) a compound which activates an HCN channel polypeptide; or

(d) a polynucleotide capable of inhibiting the expression of an HCN channel polypeptide,

for the manufacture of a medicament for treating, stroke, ischaemia, head injury, epilepsy, Alzheimer's disease, Parkinson's disease, learning or memory and attention disorders.

The present invention also provides for the use of a compound selected from:

(a) an HCN channel polypeptide, or a fragment thereof;

(b) a compound which inhibits an HCN channel polypeptide;

(c) a compound which activates an HCN channel polypeptide; or

(d) a polynucleotide capable of inhibiting the expression of an HCN channel polypeptide,

for the manufacture of a medicament for treating pain, migraine, gut disorders, in particular IBS, or sleep disorders.

The invention also relates to newly identified HCN1 polypeptides and polynucleotides. A partial HCN1 channel has previously been published (GenBank Accession number AF064876; Santoro et al PNAS (1997)94(26) pp14815-20). However a full-length human HCN1 polynucleotide sequence is given hereinbelow as SEQ ID NO:1 and the encoded polypeptide sequence as SEQ ID NO:2. The invention further relates to uses of these new polypeptides and polynucleotides.

In a first aspect, the present invention relates to the use of an HCN channel polypeptide for the manufacture of a medicament for treating, stroke, ischaemia, head injury, epilepsy, Alzheimer's disease, Parkinson's disease, learning or memory and attention disorders, pain, migraine, gut disorders, in particular IBS, or sleep disorders. Preferably such polypeptides include a human HCN channel polypeptide, in particular:

a) an isolated HCN1 polypeptide comprising an amino acid sequence having at least 95% identity to that of SEQ ID NO:2 over the entire length of SEQ ID NO:2;

b) an isolated HCN2 polypeptide comprising an amino acid sequence having at least 95% identity to that of SEQ ID NO:4, over the entire length of SEQ ID NO:4, where SEQ ID NO:4 is the sequence disclosed in GenBank Accession No: CAB42602;

c) an isolated HCN3 polypeptide characterised in that said polypeptide comprises a sequence that has at least 95% identity with the partial HCN3 sequence of SEQ ID NO:8, over the entire length of SEQ ID NO:8, where SEQ ID NO:8 is the sequence disclosed in GenBank Accession No: BAA96059; and

d) an isolated HCN4 polypeptide comprising an amino acid sequence having at least 95% identity to that of SEQ ID NO:6, over the entire length of SEQ ID NO:6, where SEQ ID NO:6 is the sequence disclosed in GenBank Accession No: CAB52754.

Such polypeptides include those comprising polypeptides having the amino acid sequence of SEQ ID NO:2, SEQ ID NO:4, SEQ ID NO:6 or SEQ ID NO:8 as well as the polypeptides of SEQ ID NO:2, SEQ ID NO:4, SEQ ID NO:6 and SEQ ID NO:8.

In addition the HCN polypeptides of the invention include variants and fragments and portions of such polypeptides in (a) to (d) that generally contain at least 30 amino acids, more preferably at least 50 amino acids, thereof.

Preferably the polypeptides of the invention are HCN1 or HCN4 polypeptides, as defined hereinabove, or fragments thereof, most preferably HCN1 polypeptides or fragments thereof.

The polypeptides of the present invention may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

The present invention also includes include variants of the aforementioned polypeptides, that is polypeptides that vary from the referents by conservative amino acid substitutions, whereby a residue is substituted by another with like characteristics. Typical such substitutions are among Ala, Val, Leu and Ile; among Ser and Thr, among the acidic residues Asp and Glu; among Asn and Gln; and among the basic residues Lys and Arg; or aromatic residues Phe and Tyr. Particularly preferred are variants in which several, 5-10, 1-5, 1-3, 1-2 or 1 amino acids are substituted, deleted, or added in any combination.

Polypeptides of the present invention can be prepared in any suitable manner. Such polypeptides include isolated naturally occurring polypeptides, recombinantly produced polypeptides, synthetically produced polypeptides, orpolypeptides produced by a combination of these methods. Means for preparing such polypeptides are well understood in the art

In a second aspect, the present invention relates to the use of an HCN channel polynucleotide for the manufacture of a medicament for treating, stroke, ischaemia, head injury, epilepsy, Alzheimer's disease, Parkinson's disease, learning or memory and attention disorders, pain, migraine, gut disorders, in particular IBS, or sleep disorders. Preferably such polynucleotides include a human HCN channel polynucleotide, in particular:

a) an isolated HCN1 polynucleotide comprising a polynucleotide sequence having at least 95% identity to that of SEQ ID NO:1 over the entire length of SEQ ID NO:1;

b) an isolated HCN2 polynucleotide comprising a polynucleotide sequence having at least 95% identity to that of SEQ ID NO:3 over the entire length of SEQ ID NO:3, where SEQ ID NO:3 is the sequence disclosed in GenBank Accession No: AJ012582;

c) an isolated HCN3 polynucleotide characterised in that said polynucleotide comprises a sequence that has at least 95% identity with the partial HCN3 polynucleotide sequence of SEQ ID NO:7, over the entire length of SEQ ID NO:7, where SEQ ID NO:7 is disclosed in GenBank Accession No: AB040968; and

d) an isolated HCN4 polynucleotide comprising a polynucleotide sequence having at least 95% identity to that of SEQ ID NO:5, over the entire length of SEQ ID NO:5, where SEQ ID NO:5 is the sequence disclosed in GenBank Accession No: AJ238850.

Such polynucleotides include those comprising polynucleotides having the sequence of SEQ ID NO:1, SEQ ID NO:3, SEQ ID NO:5 or SEQ ID NO:7 as well as the polynucleotides of SEQ ID NO:1, SEQ ID NO:3, SEQ ID NO:5 and SEQ ID NO:7.

The polynucleotide sequences encoding the polypeptides of SEQ ID NO:2, SEQ ID NO:4 SEQ ID NO:6, or SEQ ID NO:8 may be identical to the polypeptide encoding sequences contained in SEQ ID NO:1, SEQ ID NO:3, SEQ ID NO:5 or SEQ ID NO:7 respectively, or they may be sequences which, as a result of the redundancy (degeneracy) of the genetic code, also encode the aforesaid polypeptides.

In addition the HCN polynucleotides of the invention include variants and fragments and portions of such polynucleotides in (a) to (d) that generally contain at least 50 nucleotides, more preferably at least 100 nucleotides, thereof.

Preferably the polynucleotides of the invention are HCN1 or HCN4 polynucleotides, as defined hereinabove, or fragments thereof, most preferably HCN1 polynucleotides or fragments thereof.

Polypeptides and polynucleotides of the present invention are herein understood to include any splice variant of the HCN channels.

HCN channel polynucleotides of the present invention may be obtained, using standard cloning and screening techniques, from a cDNA library derived from mRNA in cells of human whole brain using techniques well established in the art (for example Sambrook et al., Molecular Cloning: A Laboratory Manual, 2nd Ed., Cold Spring Harbor Laboratory Press, Cold Spring Harbor, N.Y.(1989). Polynucleotides of the invention can also be obtained from natural sources such as genomic DNA libraries or can be synthesized using well known and commercially available techniques.

Recombinant HCN channel polypeptides of the present invention may be prepared by processes well known in the art from genetically engineered host cells comprising expression vectors comprising HCN channel encoding polynucleotides. Cell-free translation systems can also be employed to produce such proteins using RNAs derived from the DNA constructs of the present invention. Methods for expressing recombinant polypeptides are well known in the art, for example Davis et al., Basic Methods in Molecular Biology (1986) and Sambrook et al., Molecular Cloning: A Laboratory Manual, 2nd Ed., Cold Spring Harbor Laboratory Press, Cold Spring Harbor, N.Y. (1989). When the HCN polypeptide of the present invention is to be expressed for use in screening assays, it is generally preferred that the polypeptide be produced at the surface of the cell. In this event, the cells may be harvested prior to use in the screening assay.

In a further aspect, the present invention relates to the use of compounds which activate (agonists) or inhibit (antagonists) HCN polypeptides for the manufacture of a medicament for treating stroke, ischaemia, head injury, epilepsy, Alzheimer's disease, Parkinson's disease, learning or memory and attention disorders, pain, migraine, gut disorders, in particular IBS, or sleep disorders. Such compounds can be identified using screens involving HCN polypeptides. Compounds may be identified from a variety of sources, for example, cells, cell-free preparations, chemical libraries, and natural product mixtures. Such agonists or antagonists so-identified may be natural or modified HCN ligands or fragments of HCN channels etc. or may be structural or functional mimetics thereof (see Coligan et aL, Current Protocols in Immunology 1(2):Chapter 5 (1991)).

Preferably the compounds activate or inhibit HCN1 or HCN4 polypeptides, most preferably HCN1 polypeptides.

In one embodiment the screening method involves the stable transfection of a standard cell line (e.g. human embryonic kidney cells, HEK293) with an HCN channel cDNA. Thereafter, cells may be loaded with a fluorescent membrane potential dye (e.g. DiBAC, Denyer et al, 1998, 3 Drug Discovery Today; Molecular Probes, USA), and exposed to valinomycin (Molecular Probes, USA) to allow membrane hyperpolaristion by potassium extrusion and subsequent activation of hHCN1. The occurrence of Iₕ, through activation of HCN, can be readily detected by fluorescent analysis of the cells by standard imaging techniques.

In a further embodiment the screening method involves stable transfection of a standard cell line (e.g. HEK293) with an HCN1 cDNA, as well as the inward rectifying potassium channel GIRK. Thereafter, cells may be loaded with a fluorescent membrane potential dye (e.g. DiBAC, Denyer et al, 1998, 3 Drug Discovery Today; Molecular Probes, USA), and exposed to a G protein coupled receptor (GPCR) agonist to activate endogenous GPCRs (e.g., somatostatin receptors,) or stably transfected GPCRs. Stimulation of these GPCRs will activate GIRK to allow membrane hyperpolaristion by potassium extrusion and subsequent activation of HCN. The occurrence of Iₕ, through activation of HCN, can be readily detected by fluorescent analysis of the cells by standard imaging techniques.

In a still further embodiment the screening method involves the use of radiotracer assays (e.g. ³H-Choline, ¹⁴C-Guanidinium, ²²Na⁺).

In another embodiment the screening method involves the use of the Intrinsic Ion Channel Fluorescence approach (Siegel et al (1998) Neuron 19, 735-741).

In a further embodiment the screening of putative HCN channel inhibitors involves adding the compound during the fluorescent dye loading, and assessing the changes in membrane potential following addition of either valinomycin, or a GPCR agonist (e.g., somatostatin), in the presence or absence of the putative HCN channel inhibitor.

In a still further embodiment, the putative HCN channel inhibitors are identified by measuring the binding of a candidate compound to the HCN channel transfected cells or membranes bearing the channel, or a fusion protein thereof by means of a label directly or indirectly associated with the candidate compound. Alternatively, the screening method involves competition with a labeled competitor. Such labeled competitors include known HCN channel antagonists, for example ZD7288 (Tocris, UK). Further, these screening methods may test whether the candidate compound results in a signal generated by activation or inhibition of the polypeptide, using detection systems appropriate to the cells bearing the polypeptide. Inhibitors of activation are generally assayed in the presence of a known activator (e.g., an agent that causes membrane hyperpolarization as described above) and the effect on activation by the activator by the presence of the candidate compound is observed. Constitutively active polpypeptides may be employed in screening methods for inverse agonists or inhibitors, in the absence of an agonist or inhibitor, by testing whether the candidate compound results in inhibition of activation of the polypeptide. Fusion proteins, such as those made from Fc portion and HCN channel polypeptide, as herein before described, can also be used for high-throughput screening assays to identify antagonists for the polypeptide of the present invention (see D. Bennett et al., J Mol Recognition, 8:52-58 (1995); and K. Johanson et al., J Biol Chem, 270(16):9459-9471 (1995)).

The polynucleotides, polypeptides of the present invention, as well as antibodies to the polypeptides of the present invention, which may be prepared usign methods well known in the art, may also be used to configure screening methods for detecting the effect of added compounds on the production of mRNA and polypeptide in cells. For example, an ELISA assay may be constructed for measuring secreted or cell associated levels of polypeptide using monoclonal and polyclonal antibodies by standard methods known in the art. This can be used to discover agonists or antagonists from suitably manipulated cells or tissues.

Examples of potential polypeptide antagonists include antibodies or, in some cases, oligonucleotides or proteins which are closely related to the ligands, substrates, receptors, enzymes, etc., as the case may be, of the polypeptide, e.g., a fragment of the ligands, substrates, receptors, enzymes, etc.; or small molecules which bind to the polypetide of the present invention but do not elicit a response, so that the activity of the polypeptide is prevented.

It will be readily appreciated by the skilled artisan that an HCN polypeptide as defined hereinabove may also be used in a method for the structure-based design of a agonist or antagonist of the HCN channel polypeptide, by:

(a) determining in the first instance the three-dimensional structure of the HCN polypeptide;

(b) deducing the three-dimensional structure for the likely reactive or binding site(s) of an agonist or antagonist;

(c) synthesing candidate compounds that are predicted to bind to or react with the deduced binding or reactive site; and

(d) testing whether the candidate compounds are indeed agonists or antagonists.

It will be further appreciated that this will normally be an iterative process.

In a further aspect, the present invention provides methods of treating abnormal conditions related to an excess of HCN channel polypeptide activity such as, for instance, stroke, ischaemia, head injury, Alzheimer's disease, Parkinson's disease, learning or memory and attention disorders. Further the invention the provides methods of treating abnormal conditions related to an excess of HCN channel polypeptide activity such as, for instance, pain, migraine, gut disorders, in particular IBS or sleep disorders. Preferably the abnormal condition is stroke, epilepsy, Alzheimer's disease, pain or migraine.

If the disease is associated with an increased or excessive activity of the HCN polypeptide several approaches are available. One approach comprises administering to a subject in need thereof an antagonist as herein above described, optionally in combination with a pharmaceutically acceptable carrier, in an amount effective to inhibit the function of the polypeptide, such as, for example, by blocking the binding of ligands, substrates, receptors, enzymes, etc., or by inhibiting a second signal, and thereby alleviating the abnormal condition. In another approach, soluble forms of the polypeptides still capable of binding the ligand, substrate, enzymes, receptors, etc. in competition with endogenous polypeptide may be administered. Typical examples of such competitors include fragments of the HCN channel polypeptide.

In still another approach, expression of the gene encoding endogenous HCN channel polypeptide can be inhibited using expression blocking techniques. Known such techniques involve the use of antisense sequences, either internally generated or externally administered (see, for example, O'Connor, J Neurochem (1991) 56:560 in Oligodeoxynucleotides as Antisense Inhibitors of Gene Expression, CRC Press, Boca Raton, Fla. (1988)). Alternatively, oligonucleotides which form triple helices (“triplexes”) with the gene can be supplied (see, for example, Lee et aL, Nucleic Acids Res (1979) 6:3073; Cooney et al., Science (1988) 241:456; Dervan et al., Science (1991) 251:1360). These oligomers can be administered per se or the relevant oligomers can be expressed in vivo. Synthetic antisense or triplex oligonucleotides may comprise modified bases or modified backbones. Examples of the latter include methylphosphonate, phosphorothioate or peptide nucleic acid backbones. Such backbones are incorporated in the antisense or triplex oligonucleotide in order to provide protection from degradation by nucleases and are well known in the art. Antisense and triplex molecules synthesised with these or other modified backbones also form part of the present invention.

In addition, expression of the human HCN channel polypeptide may be prevented by using ribozymes specific to the human HCN channel MRNA sequence. Ribozymes are catalytically active RNAs that can be natural or synthetic (see for example Usman, N, et al., Curr. Opin. Struct. Biol (1996) 6(4), 527-33.) Synthetic ribozymes can be designed to specifically cleave HCN mRNAs at selected positions thereby preventing translation of the human HCN channel mRNAs into functional polypeptide. Ribozymes may be synthesised with a natural ribose phosphate backbone and natural bases, as normally found in RNA molecules. Alternatively the ribosymes may be synthesised with non-natural backbones to provide protection from ribonuclease degradation, for example, 2′-O-methyl RNA, and may contain modified bases.

For treating abnormal conditions or diseases related to an under-expression of the HCN channel of the invention and its activity, several approaches are also available. One approach comprises administering to a subject a therapeutically effective amount of a compound which activates an HCN channel polypeptide of the present invention, i.e., an agonist as described above, in combination with a pharmaceutically acceptable carrier, to thereby alleviate the abnormal condition. Alternatively, gene therapy may be employed to effect the endogenous production of the HCN channel by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For an overview of gene therapy, see Chapter 20, Gene Therapy and other Molecular Genetic-based Therapeutic Approaches, (and references cited therein) in Human Molecular Genetics, T Strachan and A P Read, BIOS Scientific Publishers Ltd (1996). Another approach is to administer a therapeutic amount of a polypeptide of the present invention in combination with a suitable pharmaceutical carrier.

The medicaments for use in treating the diseases mentioned hereinabove are pharmaceutical compositions comprising a therapeutically effective amount of a polypeptide, such as the soluble form of an HCN polypeptide, antagonist peptide or small molecule compound, in combination with a pharmaceutically acceptable carrier or excipient. Such carriers include, but are not limited to, saline, buffered saline, dextrose, water, glycerol, ethanol, and combinations thereof. The invention further relates to pharmaceutical packs and kits comprising one or more containers filled with one or more of the ingredients of the aforementioned compositions of the invention. Polypeptides and other compounds of the present invention may be employed alone or in conjunction with other compounds, such as therapeutic compounds.

The composition will be adapted to the route of administration, for instance by a systemic or an oral route. Preferred forms of systemic administration include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if a polypeptide or other compounds of the present invention can be formulated in an enteric or an encapsulated formulation, oral administration may also be possible. Administration of these compounds may also be topical and/or localized, in the form of salves, pastes, gels, and the like.

The dosage range required depends on the choice of peptide or other compounds of the present invention, the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

The invention further relates to an isolated HCN1 polypeptide selected from the group consisting of:

(a) an isolated polypeptide encoded by a polynucleotide comprising the sequence of SEQ ID NO:1;

(b) an isolated polypeptide comprising a polypeptide sequence having at least 95% identity to the polypeptide sequence of SEQ ID NO:2;

(c) an isolated polypeptide having at least 95% identity to the polypeptide sequence of SEQ ID NO:2; and

(d) fragments and variants of such polypeptides in (a) to (c). In a preferred embodiment the invention relates to a polypeptide comprising the polypeptide sequence of SEQ ID NO:2. In a most preferred embodiment the polypeptide is the polypeptide of SEQ IDS NO:2.

The invention also relates to an isolated polynucleotide selected from the group consisting of:

(a) an isolated polynucleotide comprising a polynucleotide sequence having at least 95% identity to the polynucleotide sequence of SEQ ID NO:1;

(b) an isolated polynucleotide having at least 95% identity to the polynucleotide of SEQ ID NO:1;

(c) an isolated polynucleotide comprising a polynucleotide sequence encoding a polypeptide sequence having at least 95% identity to the polypeptide sequence of SEQ ID NO:2;

(d) an isolated polynucleotide having a polynucleotide sequence encoding a polypeptide sequence having at least 95% identity to the polypeptide sequence of SEQ ID NO:2;

(e) an isolated polynucleotide with a nucleotide sequence of at least 100 nucleotides obtained by screening a library under stringent hybridization conditions with a labeled probe having the sequence of SEQ ID NO: 1 or a fragment thereof having at least 15 nucleotides;

(f) a polynucleotide which is the RNA equivalent of a polynucleotide of (a) to (e); or a polynucleotide sequence complementary to said isolated polynucleotide and polynucleotides that are variants and fragments of the above mentioned polynucleotides or that are complementary to above mentioned polynucleotides, over the entire length thereof.

Preferably the isolated polynucleotide is selected from the group consisiting of

(a) an isolated polynucleotide comprising the polynucleotide of SEQ ID NO:1;

(b) the isolated polynucleotide of SEQ ID NO:1;

(c) an isolated polynucleotide comprising a polynucleotide sequence encoding the polypeptide of SEQ ID NO:2; and

(d) an isolated polynucleotide encoding the polypeptide of SEQ ID NO:2.

Isolated HCN1 polynucleotides of the present invention can be obtained using standard cloning and screening techniques from a cDNA library derived from mRNA in cells of human brain (see for instance, Sambrook et al., Molecular Cloning: A Laboratory Manual, 2nd Ed., Cold Spring Harbor Laboratory Press, Cold Spring Harbor, N.Y. (1989)). Polynucleotides of the invention can also be obtained from natural sources such as genomic DNA libraries or can be synthesized using well known and commercially available techniques. Using techniques well know in the art (eg. Sambrook et al supra) the HCN1 polynucleotides can be used for the recombinant production of the polypeptides of the present invention.

Isolated HCN1 polypeptides of the present invention can also be used to devise screens for agonist and antagonist compounds for the treatment of one or more of the diseases mentioned hereinabove. Examples of such screens are outlined hereinabove.

The invention also relates to the use of HCN polynucleotides and polypeptides as diagnostic reagents. Detection of a mutated form of the gene characterised by the polynucleotide of SEQ ID NO:1, SEQ ID NO:3, SEQ ID NO:5 or SEQ ID NO:7 which is associated with a dysfunction or disease mentioned hereinabove, in particular epilepsy, stroke, Alzheimer's disease, pain or migraine, will provide a diagnostic tool that can add to, or define, a diagnosis of a disease, or susceptibility to a disease, which results from under-expression, over-expression or altered expression of the gene. Individuals carrying mutations in the gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labelled HCN polynucleotide sequences. Perfectly matched sequences can be distinguished from mismatched duplexes by RNAse digestion or by differences in melting temperatures. DNA sequence differences may also be detected by alterations in electrophoretic mobility of DNA fragments in gels, with or without denaturing agents, or by direct DNA sequencing (ee, e.g., Myers et al, Science (1985) 230:1242). Sequence changes at specific locations may also be revealed by nuclease protection assays, such as Rnase and S1 protection or the chemical cleavage method (see Cotton et al., Proc Natl Acad Sci USA (1985) 85: 4397-4401). In another embodiment, an array of oligonucleotides probes comprising HCN polynucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability (see for example: M. Chee et al., Science, Vol 274, pp 610-613 (1996)).

The diagnostic assays offer a process for diagnosing or determining a susceptibility to the diseases mentioned hereinabove, in particular epilepsy, stroke, Alzheimer's disease, pain or migraine, through detection of mutation in one or more HCN genes by the methods described. In addition, such diseases may be diagnosed by methods comprising determining from a sample derived from a subject an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, Rnase protection, Northern blotting and other hybridization methods. Assay techniques that can be used to determine levels of a protein, such as a polypeptide of the present invention, in a sample derived from a host are well-known to those of skill in the art. Such assay methods include radioimmunoassays, competitive-binding assays, Western Blot analysis and ELISA assays.

Thus in another aspect, the present invention relates to a diagonostic kit which comprises:

(a) a polynucleotide of the present invention, preferably the nucleotide sequence of SEQ ID NO: 1, SEQ ID NO:3, SEQ ID NO:5, SEQ ID NO:7 or a fragment thereof;

(b) a nucleotide sequence complementary to that of (a);

(c) a polypeptide of the present invention, preferably the polypeptide of SEQ ID NO:2, SEQ ID NO:4, SEQ ID NO:6, SEQ ID NO:8 or a fragment thereof; or

(d) an antibody to a polypeptide of the present invention, preferably to the polypeptide of SEQ ID NO:2, SEQ ID NO:4, SEQ ID NO:6 or SEQ ID NO:8.

It will be appreciated that in any such kit, (a), (b), (c) or (d) may comprise a substantial component. Such a kit will be of use in diagnosing a disease or suspectability to a disease, particularly epilepsy, stroke, Alzheimer's disease, pain or migraine amongst others.

The following definitions are provided to facilitate understanding of certain terms used frequently herein.

“Neuroprotection” is the process of rescue/saving of neurones from a substance/condition/event that would otherwise have triggered to the death/degeneration/loss of viability of the neurone.

“Excitotoxicity” is a form of neuronal cell death which is characterised by excessive release (metabolic or otherwise) of glutamate, and subsequent excessive glutamate receptor stimulation. A form of neuronal cell death which is typically prevented by the administration of glutamate receptor antagonists, such as MK-801 or AP5.

“HCN channel” is a selective sodium/potassium permeable cation channel that is activated by membrane hyperpolarisation and modulated by cAMP and cGMP. Activation of HCN channels will typically lead to the development of an inward current carried by sodium/potassium which causes depolarisation of the membrane potential. “An HCN channel” as used herein can refer to one or more of the HCN1, HCN2, HCN3 or HCN4 channels in any combination, either in a homooligomeric or a heterooligomeric arrangement.

“Fragment” of a polypeptide sequence refers to a polypeptide sequence that is shorter than the reference sequence but that retains essentially the same biological function or activity as the reference polypeptide. “Fragment” of a polynucleotide sequence refers to a polynucloetide sequence that is shorter than the reference sequence of SEQ ID NO:1 or SEQ ID NO:3.

“Fusion protein” refers to a protein encoded by two, often unrelated, fused genes or fragments thereof. In one example, EP-A-0 464 discloses fusion proteins comprising various portions of constant region of immunogiobulin molecules together with another human protein or part thereof. In many cases, employing an immunoglobulin Fc region as a part of a fusion protein is advantageous for use in therapy and diagnosis resulting in, for example, improved pharmacokinetic properties [see, e.g., EP-A 0232 262]. On the other hand, for some uses, it would be desirable to be able to delete the Fc part after the fusion protein has been expressed, detected, and purified.

“Identity” reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared. For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and similarity of two or more sequences are well known in the art. Thus for instance, programs available in the Wisconsin Sequence Analysis Package, version 9.1 (Devereux J., et al, Nucleic Acids Res, 12, 387-395, 1984, available from Genetics Computer Group, Madison, Wis., USA), for example the programs BESTFIT and GAP, may be used to determine the % identity between two polynucleotides and the % identity and the % similarity between two polypeptide sequences. BESTFIT uses the “local homology” algorithm of Smith and Waterman (J. Mol. Biol., 147:195-197, 1981, Advances in Applied Mathematics, 2, 482-489, 1981) and finds the best single region of similarity between two sequences. BESTFIT is more suited to comparing two polynucleotide or two polypeptide sequences that are dissimilar in length, the program assuming that the shorter sequence represents a portion of the longer. In comparison, GAP aligns two sequences, finding a “maximum similarity”, according to the algorithm of Neddleman and Wunsch (J. Mol Biol., 48, 443-453, 1970). GAP is more suited to comparing sequences that are approximately the same length and an alignment is expected over the entire length. Preferably, the parameters “Gap Weight” and “Length Weight” used in each program are 50 and 3, for polynucleotide sequences and 12 and 4 for polypeptide sequences, respectively. Preferably, % identities and similarities are determined when the two sequences being compared are optimally aligned.

Other programs for determining identity and/or similarity between sequences are also known in the art, for instance the BLAST family of programs (Altschul S. F., et al., J. Mol. Biol., 215, 403-410, 1990, Altschul S. F., et al., Nucleic Acids Res., 25:389-3402, 1997, available from the National Center for Biotechnology Information (NCBI), Bethesda, Md., USA and accessible through the home page of the NCBI at www.ncbi.nlm.nih.gov) and FASTA (Pearson W R, Methods in Enzymology, 183: 63-99 (1990); Pearson W R and Lipman D. J., Proc Nat Acad Sci USA, 85: 2444-2448 (1988) (available as part of the Wisconsin Sequence Analysis Package).

Preferably, the BLOSUM62 amino acid substitution matrix (Henikoff S. and Henikoff J. G., Proc. Nat. Acad Sci. USA, 89: 10915-10919 (1992)) is used in polypeptide sequence comparisons including where nucleotide sequences are first translated into amino acid sequences before comparison.

Preferably, the program BESTFIT is used to determine the % identity of a query polynucleotide or a polypeptide sequence with respect to a polynucleotide or a polypeptide sequence of the present invention, the query and the reference sequence being optimally aligned and the parameters of the program set at the default value, as hereinbefore described. Alternatively, for instance, for the purposes of interpreting the scope of a claim including mention of a “% identity” to a reference polynucleotide, a polynucleotide sequence having, for example, at least 95% identity to a reference polynucleotide sequence is identical to the reference sequence except that the polynucleotide sequence may include up to five point mutations per each 100 nucleotides of the reference sequence. Such point mutations are selected from the group consisting of at least one nucleotide deletion, substitution, including transition and transversion, or insertion. These point mutations may occur at the 5′ or 3′ terminal positions of the reference polynucleotide sequence or anywhere between these terminal positions, interspersed either individually among the nucleotides in the reference sequence or in one or more contiguous groups within the reference sequence. In other words, to obtain a polynucleotide sequence having at least 95% identity to a reference polynucleotide sequence, up to 5% of the nucleotides of the in the reference sequence may be deleted, substituted or inserted, or any combination thereof, as herein before described. The same applies mutatis mutandis for other % identities such as 96%, 97%, 98%, 99% and 100%.

For the purposes of interpreting the scope of a claim including mention of a “% identity” to a reference polypeptide, a polypeptide sequence having, for example, at least 95% identity to a reference polypeptide sequence is identical to the reference sequence except that the polypeptide sequence may include up to five point mutations per each 100 amino acids of the reference sequence. Such point mutations are selected from the group consisting of at least one amino acid deletion, substitution, including conservative and non-conservative substitution, or insertion. These point mutations may occur at the amino- or carboxy-terminal positions of the reference polypeptide sequence or anywhere between these terminal positions, interspersed either individually among the amino acids in the reference sequence or in one or more contiguous groups within the reference sequence. In other words, to obtain a sequence polypeptide sequence having at least 95% identity to a reference polypeptide sequence, up to 5% of the amino acids of the in the reference sequence may be deleted, substituted or inserted, or any combination thereof, as hereinbefore described. The same applies mutatis mutandis for other % identities such as 96%, 97%, 98%, 99%, and 100%.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 95, 97 or 100% identity to the reference sequence of SEQ ID NO:1, wherein said polynucleotide sequence may be identical to the reference sequence of SEQ ID NO:1 or may include up to a certain integer number of nucleotide alterations as compared to the reference sequence, wherein said alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein said number of nucleotide alterations is determined by multiplying the total number of nucleotides in SEQ ID NO:1 by the integer defining the percent identity divided by 100 and then subtracting that product from said total number of nucleotides in SEQ ID NO:1, or:

nₙ≦xₙ−(xₙ·y),

wherein nₙ is the number of nucleotide alterations, xₙ is the total number of nucleotides in SEQ ID NO:1, y is 0.95 for 95%, 0.97 for 97% or 1.00 for 100%, and · is the symbol for the multiplication operator, and wherein any non-integer product of xₙ and y is rounded down to the nearest integer prior to subtracting it from xₙ. Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 95, 97 or 100% identity to a polypeptide reference sequence of SEQ ID NO:2, wherein said polypeptide sequence may be identical to the reference sequence of SEQ ID NO:2 or may include up to a certain integer number of amino acid alterations as compared to the reference sequence, wherein said alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein said number of amino acid alterations is determined by multiplying the total number of amino acids in SEQ ID NO:2 by the integer defining the percent identity divided by 100 and then subtracting that product from said total number of amino acids in SEQ ID NO:2, or:

nₐ≦xₐ−(xₐ·y),

wherein nₐ is the number of amino acid alterations, xₐ is the total number of amino acids in SEQ ID NO:2, y is 0.95 for 95%, 0.97 for 97% or 1.00 for 100%, and · is the symbol for the multiplication operator, and wherein any non-integer product of xₐ and y is rounded down to the nearest integer prior to subtracting it from xₐ.

“Isolated” means altered “by the hand of man” from its natural state, ie., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Splice Variant” as used herein refers to cDNA molecules produced from RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one MRNA molecule each of that may encode different amino acid sequences. The term splice variant also refers to the proteins encoded by the above cDNA molecules.

“Polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs comprising one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may comprise amino acids other than the 20 gene-encoded amino acids. “Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may comprise many types of modifications. Polypeptides may be branched as a result of ubiquitination, and they may be cyclic, with or without branching. Cyclic, branched and branched cyclic polypeptides may result from post-translation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (see, for instance, PROTEINS—STRUCTURE AND MOLECULAR PROPERTIES, 2nd Ed., T. E. Creighton, W. H. Freeman and Company, New York, 1993; Wold, F., Post-translational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol.(1990) 182:626-646 and Rattan, et al., “Protein Synthesis: Post-translational Modifications and Aging”, Ann NY Acad Sci (1992) 663:48-62).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

All publications including, but not limited to, patents and patent applications, cited in this specification or to which this patent application claims priority, are herein incorporated by reference as if each individual publication were specifically and individually indicated to be incorporated by reference herein as though fully set forth.

### EXAMPLES

Example 1

### Human Tissue Localisation of HCN1 mRNA

TaqMan quantitative RT-PCR was carried out as previously described (Medhurst et al.(1999) Br. J. Pharmacol. 128:627-636. Human polyA+ mRNA samples were obtained from Clontech. OligodT-primed cDNA synthesis was performed in triplicate using 200 ng human polyA+ mRNA and Superscript II reverse transcriptase according to manufacturers instructions (Life Technologies). TaqMan PCR assays were performed on cDNA samples or genomic DNA standards in 96-well optical plates on an ABI Prism 7700 Sequence Detection system (PE Applied Biosystems) according to manufacturers instructions. The primer and probe sequences were as follows:

For human HCN1:

For human HCN1:

| | | |
|---|---|---|
| sense | 5′-GGCCATGCTGACCAAGCT | SEQ ID NO:9 |
| anti- | 5′-GTGCCTTCGCGGATGATG | SEQ ID NO:10 |
| sense | | |
| probe | 5′-TCACCCGGCTGGAAGACCTCGA | SEQ ID NO:11 |

For human GAPDH:

| | | |
|---|---|---|
| sense | 5′-TGAGACAGCAGATAGAGCCAAGC | SEQ ID NO:12 |
| anti- | 5′-TCCCTGCCAATTTGACATCTTC | SEQ ID NO:13 |
| sense | | |
| probe | 5′-CATCACCATTGGCAATGAGCGGTTCC | SEQ ID NO:14 |

Data were analysed using the relative standard curve method with each sample being normalised to GAPDH to correct for differences in RNA quality and quantity (Medhurst et al (1999) supra).

The human HCN1 channel was found to have a distinct tissue distribution, being found in the hypothalamus, the olfactory bulb, neocortex, piriform cortex, hippocampal pyrimidal cell layers CA1-CA3, thalamus and the cerebellum (molecular layer, Purkinje cells, granule cells).

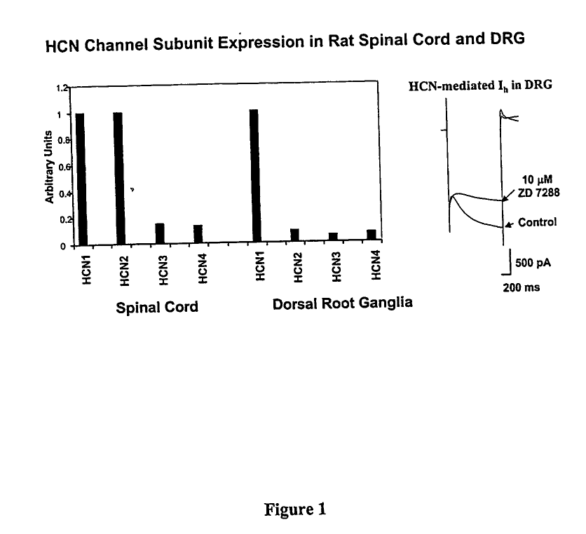

Further localisation studies have showed that HCN1, HCN2, HCN3 and HCN4 mRNAs can all be detected in rat spinal cord and dorsal root ganglia. HCN1 and HCN2 mRNA are expressed in spinal cord at higher levels than HCN3 and HCN4 (approximately 5-fold), whilst in dorsal root ganglia HCN1 is expressed at higher quantities than either HCN2, HCN3 or HCN4 (approximately 10 fold) (FIG. 1).

### Example 2

### Oxygen/Glucose Deprivation of Hippocampal Organotypic Slice Cultures

Organotypic hippocampal slice cultures were prepared using the method of Stoppini et al (1991) J. Neurosci Methods 37, 173-182. In brief, hippocampi were isolated from 8-10 day old Lister Hooded rat pups and sliced into 400 μm transverse sections using a McIlwain tissue chopper. Slices were placed into ice cold Geys balanced salt solution (supplemented with 5 mg/ml glucose and 1.5% fingizone (GIBCO/BRL). From here slices were transferred onto semiporous membranes (Millipore) at the interface of a support medium comprising 50% minimal essential medium (MEM, ICN), 25% Hanks' balanced salt solution (ICN), 25% heat inactivated horse serum (GIBCO/BRL) supplemented with 5 mg/ml glucose, 1 mM glutamine and 1.5% fungizone. Slices were maintained in this configuration in a 5% CO₂ incubator maintained at 37° C. for 14 days with the support medium being changed every 3 days.

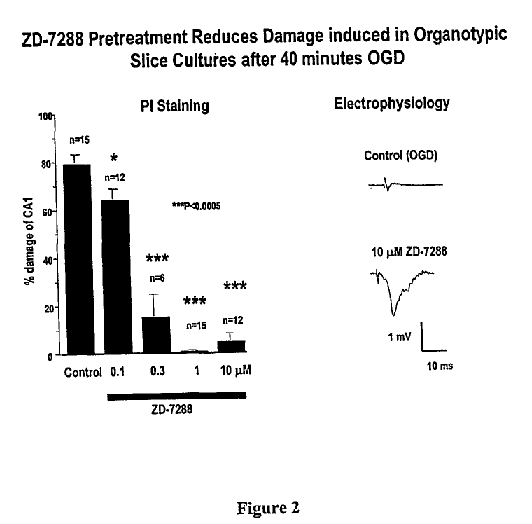

On the day of the insult organotypic slice cultures were intially placed in serum-free medium containing 5 μg/ml of the fluorescent exclusion dye propidium iodide (PI, Molecular Probes) and imaged using a Zeiss Axiovert 135 microscope and Photonic Science CCD Camera. Any cultures which exhibited PI fluorescence were discarded. The remaining slices were then subjected to an anoxic insult either in the absence or presence of ZD7288. The anoxic insult was induced by replacing the normal culture medium with serum free medium which had previously been saturated with 95% N₂/5% CO₂. The cultures were then placed into an airtight incubation chamber equipped with inlet and outlet valves and 95% N₂/5% CO2 blown through the chamber for 40 min to ensure maximal removal of oxygen. Following hypoxia cultures were transferred to normal serum free medium and placed in a 5% CO₂ incubator at 37° C. for 24 hrs before being assessed for neuronal damage using the PI staining protocol described above. Quantification of the extent of damage in the hippocampal CA1 region was assessed using IMAGE 1.55 analysis software (Wayne Rasband, NIH). A summary of the results is provided in Table 1 and FIG. 2.

TABLE 1

| TREATMENT | DAMAGE (% Cell Loss) |
|---|---|
| INSULT | 79.2 ± 4.1 |
| INSULT + ZD-7288 (10 μM)¹ | 4.58 ± 3.09 |
| INSULT + ZD-7288 (1 μM)¹ | 0.72 ± 0.44 |
| INSULT + ZD-7288 (300 nM)¹ | 15.12 ± 9.54 |
| INSULT + ZD-7288 (100 nM)¹ | 64.01 ± 4.66 |
| INSULT | 55.64 ± 17.72 |
| INSULT + ZD-7288 (100 μM)² | 12.50 ± 8.24 |
| INSULT + MK-801 (10 μM)² | 34.90 ± 15.99 |

¹Indicates that compounds were added for 60 minutes before, during, and for 24 h after the insult.
²Indicates that compounds were added immediately after the insult and for the 24 h recovery period only.

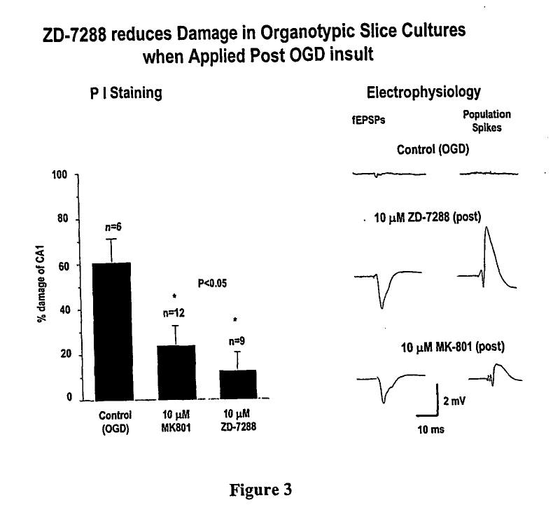

FIG. 3 shows that when ZD7288 was applied immediately after oxygen glucose deprivation neuroprotection was still observed. FIG. 3 also shows that in ZD7288 treated slices subjected to OGD it is possible to evoke normal electrophysiological responses in stratum pyramidale and stratum radiatum of area CA1 using single-shock electrical stimulation in stratum radiatum.

### Example 3

### Excitotoxicity in Dispersed Primary Hippocampal Cultures

Primary Hippocampal Cell cultures were prepared as follows. Hippocampi were isolated from embryonic Sprague Dawley rats (gestational age 17.5 days; Charles River), incubated with 0.08% (w/v) trypsin, and dissociated in Neurobasal medium containing 10% heat-inactivated fetal calf serum (Skaper et al.(1990) Methods in Neurosciences, Vol. 2 (Conn P. M., ed), pp. 17-33 Academic Press, San Diego). Cells were pelleted by centrifugation (200 g, 5 min) and resuspended in Neurobasal medium containing B27 supplements (with antioxidants), 25 μM glutamate, 1 mM sodium pyruvate, 2 mM L-glutamine, 50 U/ml penicillin, and 50 μg/ml streptomycin. The cell suspension was plated onto poly-D-lysine (10 μg/ml) coated 48-well culture plates (Nunc), at a density of 4.5×10⁴ cells per cm². Cultures were maintained at 37° C. in a humidified atmosphere of 5% CO₂-95% air. After 5 days, one-half the medium was replaced with an equal volume of maintenance medium (plating medium but containing B27 supplements without antioxidants, and lacking glutamate). Additional medium exchanges (0.5 volume) were performed every 3-4 days thereafter. Cells were used between 14-16 days in culture. During this period, neurons developed extensive neuritic networks, and formed functional synapses

Neurotoxicity was induced as follows. Cultures were washed once with Locke's solution (pH 7.0-7.4) (Skaper et al. (1990) supra) with or without 1 mM magnesium chloride (MgCl₂). To induce sub-maximal neurotoxicity, cultures were exposed for 15 min at room temperature to MgCl₂-free Locke's solution, supplemented with 0.1 μM glycine and 30 μM histamine. Thereafter, cells were washed with complete Locke's solution and returned to their original culture medium for 24 h. Cytotoxicity was evident during the 24 h after the insult. Viable neurons had phase-bright somata of round-to-oval shape, with smooth, intact neurites. Neurons were considered nonviable when they exhibited neurite fragmentation and somatic swelling and vacuolation. Cell survival was quantified 24 h after the insult by a colorimetric reaction with 3-(4,5-dimethylthiazol-2-yl)-2,5-diphenyltetrazolium bromide (MTT) (Mosmann (1983) J. Immunol. Methods 65:55-63; Manthorpe et al. (1986) Dev. Brain Res. 25:191-198; Skaper et al., (1990) supra). Absolute MTT values obtained were normalized and expressed as a percentage of sham-treated sister cultures (defined as 100%). Control experiments showed that the loss of viable neurons assessed in this manner was proportional to the number of neurons damaged, as estimated by trypan blue staining.

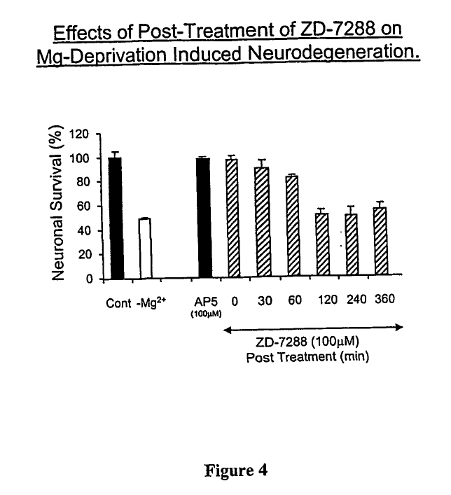

The results, given in Table 2, show that in the hippocampal neurones ZD7288 was neuroprotective (IC₅₀ for inhibition of damage of approximately 120 μM). FIG. 4 shows that the application of ZD7288 can be delayed for up to 60 minutes after the insult without loss of it's neuroprotective efficacy.

TABLE 2

| TREATMENT | NEURONAL SURVIVAL (%) |
|---|---|
| CONTROL | 100 ± 4 |
| INSULT | 46.2 ± 6.9 |
| INSULT + ZD-7288 (300 μM)¹ | 88.5 ± 2.7 |
| INSULT + ZD-7288 (100 μM)¹ | 65.9 ± 3.7 |
| INSULT + ZD-7288 (30 μM)¹ | 56.7 ± 2.0 |
| INSULT + ZD-7288 (10 μM)¹ | 48.4 ± 5.5 |
| INSULT + ZD-7288 (3 μM)¹ | 40.1 ± 8.1 |
| INSULT + ZD-7288 (1 μM)¹ | 40.6 ± 4.9 |
| INSULT + ZD-7288 (300 μM)² | 97.9 ± 9.6 |
| INSULT + ZD-7288 (100 μM)² | 79.5 ± 2.1 |
| INSULT + ZD-7288 (300 μM)³ | 101.0 ± 1.2 |
| INSULT + ZD-7288 (100 μM)³ | 78.0 ± 5.5 |
| INSULT + MK-801 (10 μM)² | 101 ± 1.2 |
| INSULT + TTX (1 μM)² | 93.4 ± 8.7 |

¹Indicates that compounds were added for 30 minutes before, during, and for 24 h after the insult.
²Indicates that compounds were added during the insult only.
³Indicates that compounds were added during the 24 h recovery period only.

### Example 4

### Electrophysiological Analysis of Cell Culture Models: Detection of Iₕ

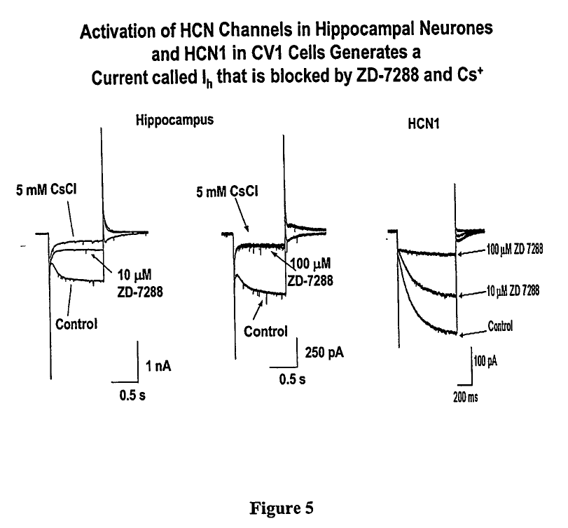

Electrophysiological analysis of neurones from both culture models, using the whole-cell patch-clamp technique, revealed the presence of Iₕ (FIG. 5). This current resembled that evoked in Cv1 cells expressing HCN1 (FIG. 5). Thus the current was activated under voltage-clamp recording conditions by hyperpolarizing steps (1 s in duration) from a holding potential of −50 mV. Succesive hyperpolarizing steps were increased in magnitude, in increments of 10 mV, such that the largest step hyperpolarized the cells to −120 mV. The hyperpolarization-activated current that was recorded exhibited all the the previously published kinetic and voltage dependent characteristics described for Iₕ and was inhibited by application of either ZD7288 (0.1-100 μM) or extracellular Cs⁺ (5 mM). Thus, Iₕ was almost completely blocked by the application of either 100 μM ZD7288 or 5 mM Cs⁺.

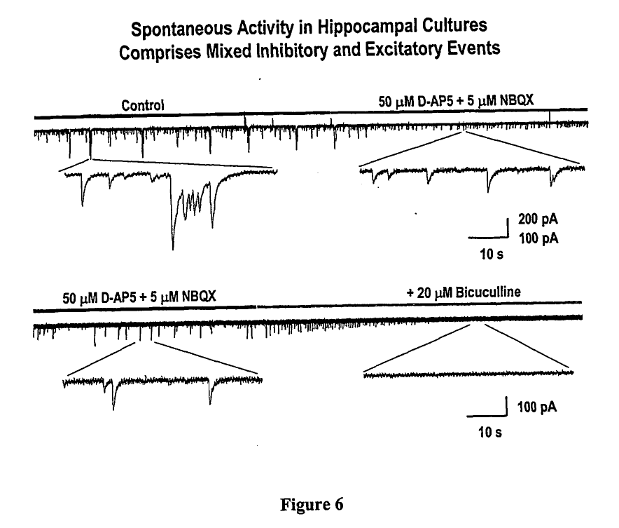

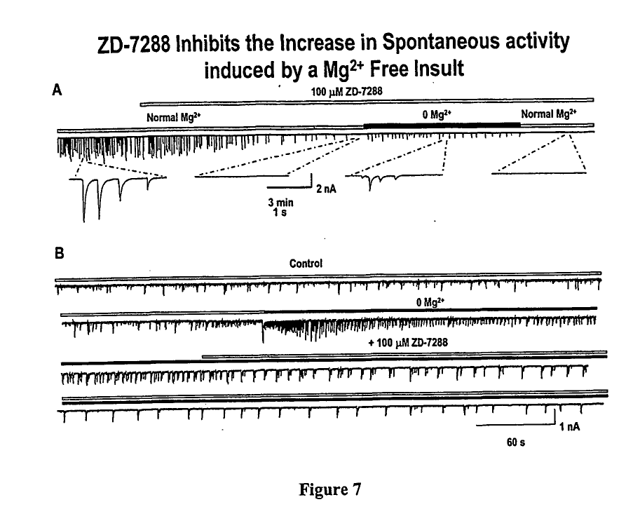

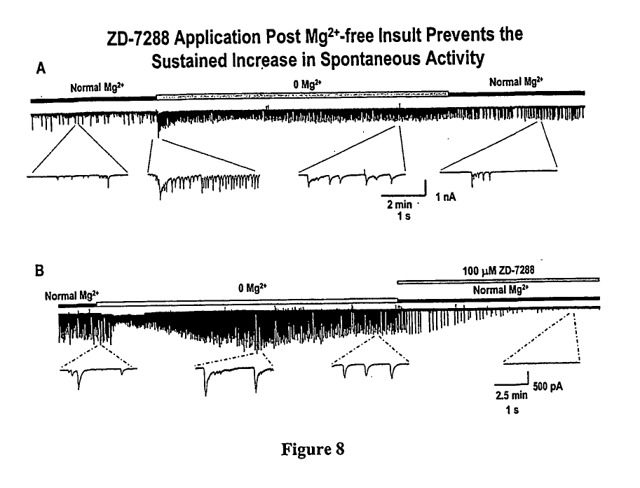

Analysis of the synaptic connectivity in hippocampal cultures revealed the presence of a high level of inhibitory GABA and excitatory glutamate receptor-mediated spontaneous activity (FIG. 6) that was greatly increased when Mg²⁺ was removed from, and glycine/histamine simultaneously added to, the bathing medium (FIG. 7). When Mg²⁺ containing medium was reinstated 15 mins later there was a sustained increase in spontaneous activity above that recorded prior to the Mg²⁺ free challenge. No change in the magnitude of the Iₕ current was recorded during or after the Mg²⁺ free challenge. However, ZD7288 (100 μM) induced a membrane potential hyperpolarization/outward current (indicative of antagonism of Iₕ) and reduced spontaneous activity irrespective of whether it was applied before, during or after the Mg²⁺-free insult (FIGS. 7 and 8). This reduction in activity differed from that induced by the NMDA receptor antagonist AP5 in that the frequency of activity in the presence of ZD7288 was much lower, yet individual events were much larger, than that observed in AP5.

These actions of ZD7288 (100 μM) were selective in that it had no direct effects on action potential firing (i.e. activation of voltage-gated Na⁺ and K⁺ channels), voltage-gated Ca²⁺ channel activation, NMDA receptor activation or metabotropic glutamate receptor activation. These findings also support the concept that the neuroprotective action of ZD7288 was mediated through inhibition of Iₕ.

### Example 5

### Epileptiform Bursting Activity in Hippocampal Slices

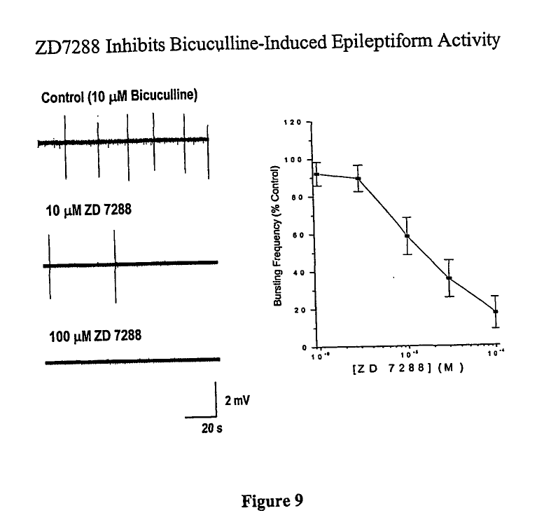

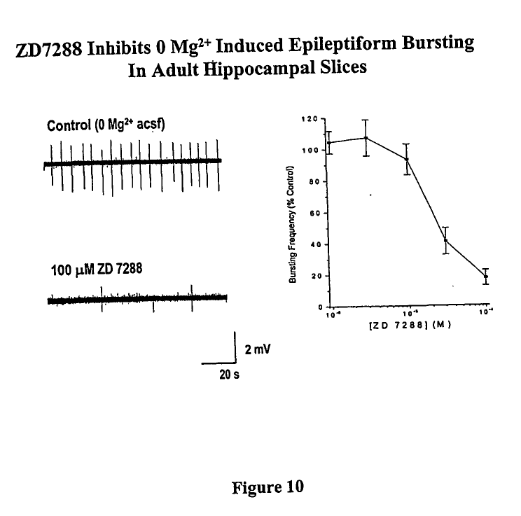

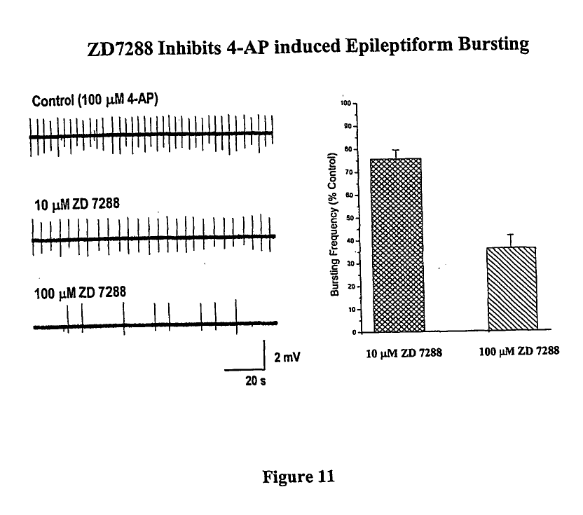

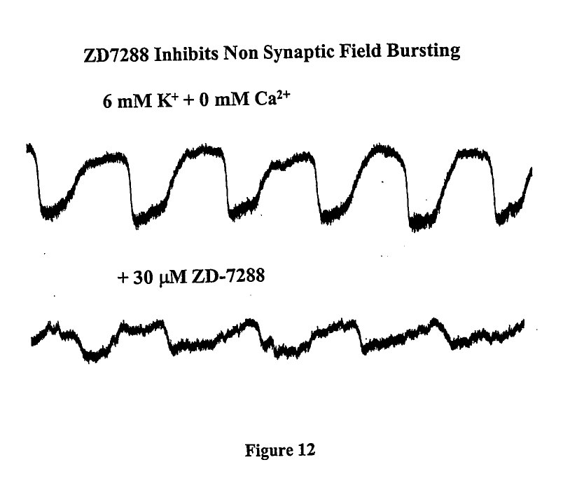

Hippocampal slices were prepared from 4-6 week old rats that had been sacrificed by cervical dislocation and subsequent decapitation in accordance with UK Home Office guidelines. The brain was removed rapidly and hippocampal slices prepared by cutting 400 μm thick horizontal sections through the whole brain minus the cerebellum using a vibroslicer (Campden Instruments, Loughborough, UK). The hippocampus from these sections was dissected free from the surrounding brain regions and the resultant hippocampal slices placed on a nylon mesh at the interface of a warmed (32-34° C.), perfusing (1-2 ml.min⁻¹) artificial cerebrospinal fluid (aCSF) and an oxygen-enriched (95% O₂, 5% CO₂), humidified atmosphere. The standard perfusion medium comprised (mM): NaCl, 124; KCl, 3; NaHCO₃, 26; NaH₂PO₄, 1.25; CaCl₂, 2; MgSO₄, 1; D-glucose, 10; and was bubbled with 95% O₂, 5% CO₂. Extracellular field potential recordings were made using glass microelectrodes (24 MΩ) filled with aCSF placed in stratum pyramidale in area CA3. Spontaneous epileptifortn activity was induced by (1) disinhibiting slices using bath application of the GABA_{A} receptor antagonist bicuculline at 10 μM (FIG. 9), (2) enhancing NMDA receptor-mediated activity by removing extracellular Mg²⁺ (FIG. 10) and (3) increasing neuronal excitability using the K⁺ channel blocker 4-aminopyridine (4-AP) (FIG. 11). Non synaptic field bursting activity (that is relevant to both epilepsy and migraine) was induced by removal of extracellular Ca²⁺ and elevation of extracellular K⁺ from 3 mM to 6-8 mM (FIG. 12). ZD7288 was deemed to have an effect on these models of neuronal hyperexcitability if it altered the frequency of events by more than 10%. Irrespective of which model was studied ZD7288 produced a concentration dependent inhibition of epileptiform bursting activity

### Example 6

### Suprachiasmatic Nucleus Function

Supraachiasmatic nucleus slices were prepared from 4-6 week old rats that had been sacrificed by cervical dislocation and subsequent decapitation in accordance with UK Home Office guidelines. The brain was removed rapidly and SCN slices prepared by cutting 400 μm thick coronal sections through the whole brain minus the cerebellum using a vibroslicer (Campden Instruments, Loughborough, UK). The resultant SCN slices were placed in a warmed (32-34° C.) submersion recording chamber perfused at 1-2 ml.min⁻¹ with an oxygen-enriched (95% O₂, 5% CO₂) artificial cerebrospinal fluid (aCSF) comprised of (mM): NaCl, 124; KCl, 3;

NaHCO₃, 26; NaH₂PO₄, 1.25; CaCl₂, 2; MgSO₄, 1; D-glucose, 10. Extracellular single unit recordings were made using glass microelectrodes (24 MΩ) filled with aCSF placed in the SCN.

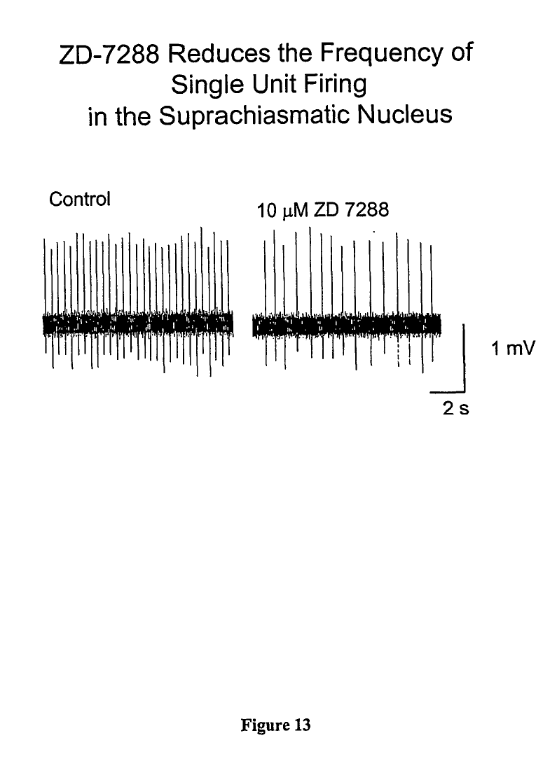

In all neurones tested ZD7288 (10-100 μM) caused a concentration dependent reduction in the frequency of single unit firing (FIG. 13).

### Example 7

### Representational Difference Analysis

The representational difference analysis (RDA) subtractive hybridisation protocol was performed on ipsilateral cortex derived from MCAO (middle cerebral artery occlusion) rats (Aspey, B. S. et al (1998) Neuropathology and Applied Neurobiology 24 p487-497) essentially as described previously (Hubank and Shatz, Nucleic Acid Research (1994), 22, 5640-5648). Briefly, 5 μg of poly A⁺ mRNA from both “tester” (normotensive rats 24 hrs following permanent MCAO) and “driver” (sham operated rats) was used to generate dscDNA. Poly A⁺ MRNA served as a template for oligodT primed reverse transcription followed by RNAse H primed second trand synthesis. Representations for both tester and driver were generated by restriction of the scDNA with Dpn II, and ligation to oligos (R-Bgl-24 and R-Bgl-12). PCR amplification with R-Bgl-24 served to generate rationalised cDNA libraries, or representations, for both tester and driver samples. The R-Bgl-24 oligo was removed from the representations by digestion with DpnII, at which point the driver representation was completed, while the tester representation was ligated to a fresh oligo pair (J-Bgl-24 and J-Bgl-12). Subtractive hybridisation was performed for 20 hrs at 67° C. in 4 μl of EEx3 buffer at a tester to driver ratio of 1: 100. Following subtraction, the hybridised cDNA was diluted in TE and cDNAs expressed at higher levels in the tester rather than driver were identified by amplification with J-Bgl-24 to generate the first difference product (DP-1). The J-Bgl-24 oligo was removed by DpnII restriction and replaced with a fresh oligo pair (N-Bgl-24 and N-Bgl-12). The N-Bgl-24 ligated cDNA served as a template for a second round of subtractive hybridisation, this time using a tester:driver ratio of 1:800. Again, differentially expressed clones were preferentially amplified from the subtracted cDNA using the tester specific oligo N-Bgl-24, to generate the second difference product (DP-2).

The subtracted library (DP-2) was restricted with DpnII and ligated into the BamHI site of the plasmid vector pcDNA3.1, before transformation into competent bacteria. Bacterial colonies were PCR screened for inserts using vector primers, and plasmid DNA extracted from positive colconies. Clones were subjected to automated sequence analysis with vector primers and identities confirmed by Blast analysis of the Genbank/EMBL databases.

Oligonucleotides (5′ to 3′):

| | | |
|---|---|---|
| R-Bg1-24 | AGCACTCTCCAGCCTCTCACCGCA | SEQ ID NO:15 |
| R-Bg1-12 | GATCTGCGGTGA | SEQ ID NO:16 |
| J-Bg1-24 | ACCGACGTCGACTATCCATGAACA | SEQ ID NO:17 |
| J-Bg1-12 | GATCTGTTCATG | SEQ ID NO:18 |
| N-Bg1-24 | AGGCAACTGTGCTATCCGAGGGAA | SEQ ID NO:19 |
| N-Bg1-12 | GATCTTCCCTCG | SEQ ID NO:20 |

Results

4 clones were identified, which rationalised into 2 contigs, that showed strong homolgy to the mouse hyperpolarising ion channel HCN4/HAC4/BCNG-3 (Accession number AF064874) in both BLASTN and BLASTX searches. Contig 545 shows 94% identity over nucleotides 65 to 301 to murine HCN4, while contig 575 shows 93% identity over nucleotides 964 to 1203 to murine HCN4. BLASTX searches showed a 93% and 100% identity for contigs 545 and 575, respectively, to murine HCN4 over the same regions.

### Example 8

### Suppressive Subtractive Hybridisation (SSH)

SSH was performed essentially as described by the manufacturers (PCR-select Clontech, Diatchenko et al. PNAS 93:6025-6030, 1996). Normotensive rats 24 hrs post permanent MCAO served as the tester, while sham-treated animals at the same timepoint were used as the driver. 2 ug of polyA⁺ mRNA was used to generate dscDNA, which was restricted with RsaI, at which point the driver was complete, while the tester was further ligated independently to two-sets of adaptors (1 and 2R). The two sets of adaptor-ligated tester cDNAs were independently hybridised with driver at a ratio of tester:driver of 1:30 for 8 hrs at 68° C., at which point the samples were combined, the tester:driver ratio increased to 1:36 and hybridisation continued for 18 hrs. Gene products expressed at higher levels in the tester than the driver were more likely to form cDNA with different oligos at either end, and are therefore immune from the suppression of PCR amplification. Differentially expressed transcripts were amplified by two round of PCR and cloned into pCDNA3.1/V5-His-TOPO (In Vitrogen) using the topoisomerase-I ligation method. Clones were subjected to automated sequence analysis with vector primers and identities confirmed by Blast analysis of the Genbank/EMBL databases.

Adaptor or oligonucleotides (5′ to 3′):

Adaptor oligonucleotides (5′ to 3′):

| | | |
|---|---|---|
| 1 | CTAATACGACTCACTATAGGGCTC | SEQ ID NO:21 |
| | GAGCGGCCGCCCGGGCAGGTACCT | |
| | GCCCGG | |
| 2R | CTAATACGACTCACTATAGGGCAG | SEQ ID NO:22 |
| | CGTGGTCGCGGCCGAGGTACCTCG | |
| | GCCG | |
| PCR primer 1 | CTAATACGACTCACTATAGGGC | SEQ ID NO:23 |
| nested primer | TCGAGCGGCCGCCCGGGCAGGT | SEQ ID NO:24 |
| 1 | | |
| primer 2R | CTAATACGACTCACTATAGGGC | SEQ ID NO:25 |
| nested primer | AGCGTGGTCGCGGCCGAGGT | SEQ ID NO:26 |
| 2R | | |

Results:

Blast analysis of the subtracted library showed that one clone, clone 39, showed high homology to the murine HCN1/HAC2/BCNG-1 gene (Accession number: AJ225123). Clone 39 shows 93% identity to mHCN1 at the nucleotide level, and 98% identity at the amino acid level over the region 2152-2418 bp of AJ225123.

1. The use of a compound selected from:
(a) an HCN channel polypeptide, or a fragment thereof;
(b) a compound which inhibits an HCN channel polypeptide;
(c) a compound which activates an HCN channel polypeptide; or
(d) a polynucleotide capable of inhibiting the expression of an HCN channel polypeptide,
for the manufacture of a medicament for treating, stroke, ischaemia, head injury, epilepsy, Alzheimer's disease, Parkinson's disease, learning or memory and attention disorders.

2. The use according to claim 1 wherein the medicament comprises an isolated polypeptide which comprises a polypeptide having at least 99% identity to the HCN channel polypeptide of SEQ ID NO:2, SEQ ID NO:4, SEQ ID NO:6 or SEQ ID NO:8.

3. The use according to claim 2 wherein the isolated polypeptide is the HCN channel polypeptide of SEQ ID NO:2, SEQ ID NO:4, SEQ ID NO:6 or SEQ ID NO:8.

4. The use according to claim 1 wherein the medicament comprises a compound which inhibits an HCN channel polypeptide.

5. The use according to claim 1 wherein the medicament comprises a compound which activates an HCN channel polypeptide.

6. The use according to claim 1 wherein the polynucleotide comprises a polynucleotide having at least 95% identity with the polynucleotide of SEQ ID NO:1, SEQ ID NO:3, SEQ ID NO:5, or SEQ ID NO:7.

7. The use according to claim 6 wherein the polynucleotide has the polynucleotide sequence of SEQ ID NO:1, SEQ ID NO:3, SEQ ID NO:5 or SEQ ID NO:7.

8. The use of a compound selected from:
(a) an HCN channel polypeptide, or a fragment thereof;
(b) a compound which inhibits an HCN channel polypeptide;
(c) a compound which activates an HCN channel polypeptide; or
(d) a polynucleotide capable of inhibiting the expression of an HCN channel polypeptide,
for the manufacture of a medicament for treating pain, gut disorders, in particular IBS, or sleep disorders.

9. The use according to claim 8 wherein the medicament comprises an isolated polypeptide which comprises a polypeptide having at least 99% identity to the HCN channel polypeptide of SEQ ID NO:2, SEQ ID NO:4, SEQ ID NO:6 or SEQ ID NO:8.

10. The use according to claim 9 wherein the isolated polypeptide is the HCN channel polypeptide of SEQ ID NO:2, SEQ ID NO:4, SEQ ID NO:6 or SEQ ID NO:8.

11. The use according to claim 8 wherein the medicament comprises a compound which inhibits an HCN channel polypeptide.

12. The use according to claim 8 wherein the medicament comprises a compound which activates an HCN channel polypeptide.

13. The use according to claim 8 wherein the polynucleotide comprises a polynucleotide having at least 95% identity with the polynucleotide of SEQ ID NO:1, SEQ ID NO:3, SEQ ID NO:5, or SEQ ID NO:7.

14. The use according to claim 13 wherein the polynucleotide has the polynucleotide sequence of SEQ ID NO: 1, SEQ ID NO:3, SEQ ID NO:5 or SEQ ID NO:7.

15. An isolated polypeptide selected from the group consisting of:
(a) an isolated polypeptide encoded by a polynucleotide comprising the sequence of SEQ ID NO:1;
(b) an isolated polypeptide comprising a polypeptide sequence having at least 95% identity to the polypeptide sequence of SEQ ID NO:2;
(c) an isolated polypeptide having at least 95% identity to the polypeptide sequence of SEQ ID NO:2; and
(d) fragments and variants of such polypeptides in (a) to (c).

16. The isolated polypeptide as claimed in claim 15 comprising the polypeptide sequence of SEQ ID NO:2.

17. The isolated polypeptide as claimed in claim 15 which is the polypeptide sequence of SEQ ID NO:2.

18. An isolated polynucleotide selected from the group consisting of:
(a) an isolated polynucleotide comprising a polynucleotide sequence having at least 95% identity to the polynucleotide sequence of SEQ ID NO:1;
(b) an isolated polynucleotide having at least 95% identity to the polynucleotide of SEQ ID NO:1;
(c) an isolated polynucleotide comprising a polynucleotide sequence encoding a polypeptide sequence having at least 95% identity to the polypeptide sequence of SEQ ID NO:2;
(d) an isolated polynucleotide having a polynucleotide sequence encoding a polypeptide sequence having at least 95% identity to the polypeptide sequence of SEQ ID NO:2;
(e) an isolated polynucleotide with a nucleotide sequence of at least 100 nucleotides obtained by screening a library under stringent hybridization conditions with a labeled probe having the sequence of SEQ ID NO: 1 or a fragment thereof having at least 15 nucleotides;
(f) a polynucleotide which is the RNA equivalent of a polynucleotide of (a) to (e); or a polynucleotide sequence complementary to said isolated polynucleotide and polynucleotides that are variants and fragments of the above mentioned polynucleotides or that are complementary to above mentioned polynucleotides, over the entire length thereof.

19. An isolated polynucleotide as claimed in claim 18 selected from the group consisting of:
(a) an isolated polynucleotide comprising the polynucleotide of SEQ ID NO:1;
(b) the isolated polynucleotide of SEQ ID NO:1;
(c) an isolated polynucleotide comprising a polynucleotide sequence encoding the polypeptide of SEQ ID NO:2; and
(d) an isolated polynucleotide encoding the polypeptide of SEQ ID NO:2.

20. An expression vector comprising a polynucleotide capable of producing a polypeptide of claim 15 when said expression vector is present in a compatible host cell.

21. A recombinant host cell comprising the expression vector of claim 20 or a membrane thereof expressing the polypeptide of claim 1.


SEQUENCE LISTING (26 sequences)

SEQ ID NO: 1 - DNA, 2670 bp, Homo sapiens
  misc_feature: n=a, t, c, or g
atggaaggaggcggcaagcccaactcttcgtctaacagccgggacgatggcaacagcgtcttccccgccaaggcgtccgcgccgggcgcggggccggccgcggccgagaagcgcctgggcaccccgccggggggcggcggggccggcgcgaaggagcacggcaactccgtgtgcttcaaggtggacggcggtggcggcggtggcggcggcggcggcggcggcgaggagccggcggggggcttcgaagacgccgaggggccccggcggcagtacggcttcatgcagaggcagttcacctccatgctgcagcccggggtcaacaaattctccctccgcatgtttgggagccagaaggcggtggaaaaggagcaggaaagggttaaaactgcaggcttctggattatccacccttacagtgatttcaggttttactgggatttaataatgctcataatgatggttggaaatctagtcatcataccagttggaatcacattctttacagagcaaacaacaacaccatggattattttcaatgtggcatcagatacagttttcctattggacctgatcatgaattttaggactgggactgtcaatgaagacagttctgaaatcatcctggaccccaaagtgatcaagatgaattatttaaaaagctggtttgtggttgacttcatctcatccatcccagtggattatatctttcttattgtagaaaaaggaatggattctgaagtttacaagacagccagggcccttcgcattgtgaggtttacaaaaattctcagtctcttgcgtttattacgactttcaaggttaattagatacatacatcaatgggaagagatattccacatgacatatgatctcgccagtgcagtggtgagaatttttaatctcatcggcatgatgctgctcctgtgccactgggatggttgtcttcagttcttagtaccactactgcaggacttcccaccagattgctgggtgtctttaaatgaaatggttaatgattcttggggaaagcagtattcatacgcactcttcaaagctatgagtcacatgctgtgcattgggtatggagcccaagccccagtcagcatgtctgacctctggattaccatgctgagcatgatcgtcggggccacctgctatgccatgtttgtcggccatgccaccgctttaatccagtctctggattcttcgaggcggcagtatcaagagaagtataagcaagtggaacaatacatgtcattccataagttaccagctgatatgcgtcagaagatacatgattactatgaacacagataccaaggcaaaatctttgatgaggaaaatattctcaatgaactcaatgatcctctgagagaggagatagtcaacttcaactgtcggaaactggtggctacaatgcctttatttgctaatgcggatcctaattttgtgactgccatgctgagcaagttgagatttgaggtgtttcaacctggagattatatcatacgagaaggagccgtgggtaaaaaaatgtatttcattcaacacggtgttgctggtgtcattacaaaatccagtaaagaaatgaagctgacagatggctcttactttggagagatttgcctgctgaccaaaggacgtcgtactgccagtgttcgagctgatacatattgtcgtctttactcactttccgtggacaatttcaacgaggtcctggaggaatatccaatgatgaggagagcctttgagacagttgccattgaccgactagatcgaataggaaagaaaaattcaattcttctgcaaaagttccagaaggatctgaacactggtgttttcaacaatcaggagaacgaaatcctcaagcagattgtgaaacatgacagggagatggtgcaggcaatcgctcccatcaattatcctcaaatgacaaccctgaattccacatcgtctactacgaccccgacctcccgcatgaggacacaatctccaccggtgtacacagcgaccagcctgtctcacagcaacctgcactcccccagtcccagcacacagaccccccagccatcagccatcctgtcaccctgctcctacaccaccgcggtctgcagccctcctgtacagagccctctggccgctcgaactttccactatgcctcccccaccgcctcccagctgtcactcatgcaacagcagccgcagcagcaggtacagcagtcccagccgccgcagactcagccacagcagccgtccccgcagccacagacacctggcagctccacgccgaaaaatgaagtgcacaagagcacgcaggcgcttcacaacaccaacctgacccgggaagtcaggccattttccgcctggcagccntcgctgccccatgaggtgtccactttgatttccagacctcatcccactgtgggggagtccctggcctccatccctcaacccgtgacggcggtccccggaacgggccttcaggcagggggcaggagcactgtcccgcagcgcgtcacctttttccgacagatgtcgtcgggagccatccccccgaaccgaggagtccttccagcaccccttccaccagcagctgctcttccaagagaatcttcctcagtcttaaacacagacccagacgcagaaaagccacgatttgcttcaaattta

SEQ ID NO: 2 - protein, 890 aa, Homo sapiens
MEGGGKPNSSSNSRDDGNSVFPAKASAPGAGPAAAEKRLGTPPGGGGAGAKEHGNSVCFKVDGGGGGGGGGGGGEEPAGGFEDAEGPRRQYGFMQRQFTSMLQPGVNKFSLRMFGSQKAVEKEQERVKTAGFWIIHPYSDFRFYWDLIMLIMMVGNLVIIPVGITFFTEQTTTPWIIFNVASDTVFLLDLIMNFRTGTVNEDSSEIILDPKVIKMNYLKSWFVVDFISSIPVDYIFLIVEKGMDSEVYKTARALRIVRFTKILSLLRLLRLSRLIRYIHQWEEIFHMTYDLASAVVRIFNLIGMMLLLCHWDGCLQFLVPLLQDFPPDCWVSLNEMVNDSWGKQYSYALFKAMSHMLCIGYGAQAPVSMSDLWITMLSMIVGATCYAMFVGHATALIQSLDSSRRQYQEKYKQVEQYMSFHKLPADMRQKIHDYYEHRYQGKIFDEENILNELNDPLREEIVNFNCRKLVATMPLFANADPNFVTAMLSKLRFEVFQPGDYIIREGAVGKKMYFIQHGVAGVITKSSKEMKLTDGSYFGEICLLTKGRRTASVRADTYCRLYSLSVDNFNEVLEEYPMMRRAFETVAIDRLDRIGKKNSILLQKFQKDLNTGVFNNQENEILKQIVKHDREMVQAIAPINYPQMTTLNSTSSTTTPTSRMRTQSPPVYTATSLSHSNLHSPSPSTQTPQPSAILSPCSYTTAVCSPPVQSPLAARTFHYASPTASQLSLMQQQPQQQVQQSQPPQTQPQQPSPQPQTPGSSTPKNEVHKSTQALHNTNLTREVRPFSAWQPSLPHEVSTLISRPHPTVGESLASIPQPVTAVPGTGLQAGGRSTVPQRVTFFRQMSSGAIPPNRGVLPAPLPPAAALPRESSSVLNTDPDAEKPRFASNL

SEQ ID NO: 3 - DNA, 3459 bp, Homo sapiens
ggccggcggcggcggcggcggctccgctccgcactgcccggcgccgcctcgccatggacgcgcgcgggggcggcgggcggcccggggagagcccgggcgcgacccccgcgccggggccgccgccgccgccgccgcccgcgcccccccaacagcagccgccgccgccgccgccgcccgcgccccccccgggccccgggcccgcgcccccccagcacccgccccgggccgaggcgttgcccccggaggcggcggatgagggcggcccgcggggccggctccgcagccgcgacagctcgtgcggccgccccggcaccccgggcgcggcgagcacggccaagggcagcccgaacggcgagtgcgggcgcggcgagccgcagtgcagccccgcggggcccgagggcccggcgcgggggcccaaggtgtcgttctcgtgccgcggggcggcctcggggcccgcgccggggccggggccggcggaggaggcgggcagcgaggaggcgggcccggcgggggagccgcgcggcagccaggccagcttcatgcagcgccagttcggcgcgctcctgcagccgggcgtcaacaagttctcgctgcggatgttcggcagccagaaggccgtggagcgcgagcaggagcgcgtcaagtcggcgggggcctggatcatccacccgtacagcgacttcaggttctactgggacttcaccatgctgctgttcatggtgggaaacctcatcatcatcccagtgggcatcaccttcttcaaggatgagaccactgccccgtggatcgtgttcaacgtggtctcggacaccttcttcctcatggacctggtgttgaacttccgcaccggcattgtgatcgaggacaacacggagatcatcctggaccccgagaagatcaagaagaagtatctgcgcacgtggttcgtggtggacttcgtgtcctccatccccgtggactacatcttccttattgtggagaagggcattgactccgaggtctacaagacggcacgcgccctgcgcatcgtgcgcttcaccaagatcctcagcctcctgcggctgctgcgcctctcacgcctgatccgctacatccatcagtgggaggagatcttccacatgacctatgacctggccagcgcggtgatgaggatctgcaatctcatcagcatgatgctgctgctctgccactgggacggctgcctgcagttcctggtgcctatgctgcaggacttcccgcgcaactgctgggtgtccatcaatggcatggtgaaccactcgtggagtgaactgtactccttcgcactcttcaaggccatgagccacatgctgtgcatcgggtacggccggcaggcgcccgagagcatgacggacatctggctgaccatgctcagcatgattgtgggtgccacctgctacgccatgttcatcggccacgccactgccctcatccagtcgctggactcctcgcggcgccagtaccaggagaagtacaagcaggtggagcagtacatgtccttccacaagctgccagctgacttccgccagaagatccacgactactatgagcaccgttaccagggcaagatgtttgacgaggacagcatcctgggcgagctcaacgggcccctgcgggaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcggggagatctgcctgctcacccggggccgccgcacggcgagcgtgcgggccgacacctactgccgcctctattcgctgagcgtggacaacttcaacgaggtgctggaggagtaccccatgatgcggcgcgccttcgagacggtggccatcgaccgcctggaccgcatcggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttgaggcccacgcccgctgcccgggccgccgcgcccagcccggaccgcagggactcggcctcacccggcgccgccggcggcctggacccccaggactccgcgcgctcgcgcctctcgtccaacttgtgaccctcgccgaccgccccgcgggcccaggcgggccaggggcggggccgtcatccagaccaaagccatgccattgcgctgccccggccgccagtccgcccagaagccatagacgagacgtaggtagccgtagttggacggacgggcagggccggcggggcagccccctccgcgcccccggccgtcccccctcatcgccccgcgcccacccccatcgcccctgcccccggcggcggcctcgcgtgcgagggggctcccttcacctcggtgcctcagttcccccagctgtaagacagggacggggcggcccagtggctgagaggagccggctgtggagccccgcccgccccccaccctctaggtggcccccgtccgaggaggatcgttttctaagtgcaatacttggcccgccggcttcccgctgcccccatcgcgctcacgcaataaccggcccggcccccgtccgcgcgcgtcccccggtgacctcggggagcagcaccccgcctccctccagcactggcaccgaggggcaggcctggctgcgcagggcgcgggggggaggctggggtcccgccgccgtgatgaatgtactgacgagccgaggcagcagtgcccccaccgtggccccccacgccccattaacccccacacccccattccgcgcaataaacgacagcattggcgccaaaaaaaaaaaaaaaaaaaaaaaaaaaaaaaaaaaaaaaaaaaaaaaaaaaaa

SEQ ID NO: 4 - protein, 889 aa, Homo sapiens
MDARGGGGRPGESPGATPAPGPPPPPPPAPPQQQPPPPPPPAPPPGPGPAPPQHPPRAEALPPEAADEGGPRGRLRSRDSSCGRPGTPGAASTAKGSPNGECGRGEPQCSPAGPEGPARGPKVSFSCRGAASGPAPGPGPAEEAGSEEAGPAGEPRGSQASFMQRQFGALLQPGVNKFSLRMFGSQKAVEREQERVKSAGAWIIHPYSDFRFYWDFTMLLFMVGNLIIIPVGITFFKDETTAPWIVFNVVSDTFFLMDLVLNFRTGIVIEDNTEIILDPEKIKKKYLRTWFVVDFVSSIPVDYIFLIVEKGIDSEVYKTARALRIVRFTKILSLLRLLRLSRLIRYIHQWEEIFHMTYDLASAVMRICNLISMMLLLCHWDGCLQFLVPMLQDFPRNCWVSINGMVNHSWSELYSFALFKAMSHMLCIGYGRQAPESMTDIWLTMLSMIVGATCYAMFIGHATALIQSLDSSRRQYQEKYKQVEQYMSFHKLPADFRQKIHDYYEHRYQGKMFDEDSILGELNGPLREEIVNFNCRKLVASMPLFANADPNFVTAMLTKLKFEVFQPGDYIIREGTIGKKMYFIQHGVVSVLTKGNKEMKLSDGSYFGEICLLTRGRRTASVRADTYCRLYSLSVDNFNEVLEEYPMMRRAFETVAIDRLDRIGKKNSILLHKVQHDLNSGVFNNQENAIIQEIVKYDREMVQQAELGQRVGLFPPPPPPPQVTSAIATLQQAAAMSFCPQVARPLVGPLALGSPRLVRRPPPGPAPAAASPGPPPPASPPGAPASPRAPRTSPYGGLPAAPLAGPALPARRLSRASRPLSASQPSLPHGAPGPAASTRPASSSTPRLRPTPAARAAAPSPDRRDSASPGAAGGLDPQDSARSRLSSNL

SEQ ID NO: 5 - DNA, 4751 bp, Homo sapiens
tcgacaaaaatgccagggaaaggcgagcccagagcttggtgatggagaaattgggaagccaccccccacccttcaatcttaggatggggaattcgcaactgaagccggagcttcagacttggggcgcactcccagcttagcccaggaaagagatttaagggcgcagcagtgtggatacctctcaccccggccccgaaggtctagcgagggtctaacctgggccccttgccaggcccgccccccgcccctttccagcccccggcccgtgcgccgctgcccctttaagaagcccaggtaggcaggcccggctgctggagccgctcctatggcaacccgcgagctgcggcggcttcatgaatattccggggcgcgggagcccgagcgctgccggagggcgcttcgggggaggcggccgctgatgtaagcccggcgggtcgctgggctccgctcggttgcggcgggagccccgggacgggccggacgggccggggcagaggaggcgaggcgagctcgcgggtggccagccacaaagcccgggcggcgagacagacggacagccagccctcccgcgggacgcacgcccgggacccgcgcgggccgtgcgctctgcactccggagcggttccctgagcgccgcggccgcagagcctctccggccggcgcccattgttccccgcgggggcggggcgcctggagccgggcggcgcgccgcccctgaacgccagagggagggagggaggcaagaagggagcgcggggtccccgcgcccagccgggcccgggaggaggtgtagcgcggcgagcccggggactcggagcgggactaggatcctccccgcggcgcgcagcctgcccaagcatgggcgcctgaggctgcccccacgccggcggcaaaggacgcgtccccacgggcggactgaccggcgggcggacctggagcccgtccgcggcgccgcgctcctgcccccggcccggtccgaccccggcccctggcgccatggacaagctgccgccgtccatgcgcaagcggctctacagcctcccgcagcaggtgggggccaaggcgtggatcatggacgaggaagaggacgccgaggaggagggggccgggggccgccaagaccccagccgcaggagcatccggctgcggccactgccctcgccctccccctcggcggccgcgggtggcacggagtcccggagctcggccctcggggcagcggacagcgaagggccggcccgcggcgcgggcaagtccagcacgaacggcgactgcaggcgcttccgcgggagcctggcctcgctgggcagccggggcggcggcacgggcggcacggggagcggcagcagtcacggacacctgcatgactccgcggaggagcggcggctcatcgccgagggcgacgcgtcccccggcgaggacaggacgcccccaggcctggcggccgagcccgagcgccccggcgcctcggcgcagcccgcagcctcgccgccgccgccccagcagccaccgcagccggcctccgcctcctgcgagcagccctcggtggacaccgctatcaaagtggagggaggcgcggctgccggcgaccagatcctcccggaggccgaggtgcgcctgggccaggccggcttcatgcagcgccagttcggggccatgctccaacccggggtcaacaaattctccctaaggatgttcggcagccagaaagccgtggagcgcgaacaggagagggtcaagtcggccggattttggattatccacccctacagtgacttcagattttactgggacctgaccatgctgctgctgatggtgggaaacctgattatcattcctgtgggcatcaccttcttcaaggatgagaacaccacaccctggattgtcttcaatgtggtgtcagacacattcttcctcatcgacttggtcctcaacttccgcacagggatcgtggtggaggacaacacagagatcatcctggacccgcagcggattaaaatgaagtacctgaaaagctggttcatggtagatttcatttcctccatccccgtggactacatcttcctcattgtggagacacgcatcgactcggaggtctacaagactgcccgggccctgcgcattgtccgcttcacgaagatcctcagcctcttacgcctgttacgcctctcccgcctcattcgatatattcaccagtgggaagagatcttccacatgacctacgacctggccagcgccgtggtgcgcatcgtgaacctcatcggcatgatgctcctgctctgccactgggacggctgcctgcagttcctggtacccatgctacaggacttccctgacgactgctgggtgtccatcaacaacatggtgaacaactcctgggggaagcagtactcctacgcgctcttcaaggccatgagccacatgctgtgcatcggctacgggcggcaggcgcccgtgggcatgtccgacgtctggctcaccatgctcagcatgatcgtgggtgccacctgctacgccatgttcattggccacgccactgccctcatccagtccctggactcctcccggcgccagtaccaggaaaagtacaagcaggtggagcagtacatgtcctttcacaagctcccgcccgacacccggcagcgcatccacgactactacgagcaccgctaccagggcaagatgttcgacgaggagagcatcctgggcgagctaagcgagcccctgcgggaggagatcatcaactttaactgtcggaagctggtggcctccatgccactgtttgccaatgcggaccccaacttcgtgacgtccatgctgaccaagctgcgtttcgaggtcttccagcctggggactacatcatccgggaaggcaccattggcaagaagatgtacttcatccagcatggcgtggtcagcgtgctcaccaagggcaacaaggagaccaagctggccgacggctcctactttggagagatctgcctgctgacccggggccggcgcacagccagcgtgagggccgacacctactgccgcctctactcgctgagcgtggacaacttcaatgaggtgctggaggagtaccccatgatgcgaagggccttcgagaccgtggcgctggaccgcctggaccgcattggcaagaagaactccatcctcctccacaaagtccagcacgacctcaactccggcgtcttcaactaccaggagaatgagatcatccagcagattgtgcagcatgaccgggagatggcccactgcgcgcaccgcgtccaggctgctgcctctgccaccccaacccccacgcccgtcatctggaccccgctgatccaggcaccactgcaggctgccgctgccaccacttctgtggccatagccctcacccaccaccctcgcctgcctgctgccatcttccgccctcccccaggatctgggctgggcaacctcggtgccgggcagacgccaaggcacctgaaacggctgcagtccctgatcccttctgcgctgggctccgcctcgcccgccagcagcccgtcccaggtggacacaccgtcttcatcctccttccacatccaacagctggctggattctctgcccccgctggactgagcccactcctgccctcatccagctcctccccaccccccggggcctgtggctccccctcggctcccacaccatcagctggcgtagccgccaccaccatagccgggtttggccacttccacaaggcgctgggtggctccctgtcctcctccgactctcccctgctcaccccgctgcagccaggcgcccgctccccgcaggctgcccagccatctcccgcgccacccggggcccggggaggcctgggactcccggagcacttcctgccacccccaccctcatccagatccccgtcatctagccccgggcagctgggccagcctcccggggagttgtccctaggtctggccactggcccactgagcacgccagagacacccccacggcagcctgagccgccgtcccttgtggcaggggcctctgggggggcttcccctgtaggctttactccccgaggaggtctcagcccccctggccacagcccaggccccccaagaaccttcccgagtgccccgccccgggcctctggctcccacggatccttgctcctgccacctgcatccagccccccaccaccccaggtcccccagcgccggggcacacccccgctcacccccggccgcctcacccaggacctcaagctcatctccgcgtctcagccagccctgcctcaggacggggcgcagactctccgcagagcctccccgcactcctcaggggagtccatggctgccttcccgctcttccccagggctgggggtggcagcgggggcagtgggagcagcgggggcctcggtccccctgggaggccctatggtgccatccccggccagcacgtcactctgcctcggaagacatcctcaggttctttgccaccccctctgtctttgtttggggcaagagccacctcttctggggggccccctctgactgctggaccccagagggaacctggggccaggcctgagccagtgcgctccaaactgccgtccaatctatgagctgggcccttccttccctcttctttcttcttttctctcccttccttcttccttcaggtttaactgtgattaggagatataccaataacagtaataattatttaaaaaaccacacacaccagaaaaacaaaagacagcagaaa

SEQ ID NO: 6 - protein, 1203 aa, Homo sapiens [sequence omitted]

SEQ ID NO: 7 - DNA, 3496 bp, Homo sapiens
gaaatcgagcaggagcgggtgaagtcagcgggggcctggatcatccacccctacagcgacttccggttttactgggacctgatcatgctgctgctgatggtggggaacctcatcgtcctgcctgtgggcatcaccttcttcaaggaggagaactccccgccttggatcgtcttcaacgtattgtctgatactttcttcctactggatctggtgctcaacttccgaacgggcatcgtggtggaggagggtgctgagatcctgctggcaccgcgggccatccgcacgcgctacctgcgcacctggttcctggttgacctcatctcttctatccctgtggattacatcttcctagtggtggagctggagccacggttggacgctgaggtctacaaaacggcacgggccctacgcatcgttcgcttcaccaagatcctaagcctgctgaggctgctccgcctctcccgcctcatccgctacatacaccagtgggaggagatctttcacatgacctatgacctggccagtgctgtggttcgcatcttcaacctcattgggatgatgctgctgctatgtcactgggatggctgtctgcagttcctggtgcccatgctgcaggacttccctcccgactgctgggtctccatcaaccacatggtgaaccactcgtggggccgccagtattcccatgccctgttcaaggccatgagccacatgctgtgcattggctatgggcagcaggcacctgtaggcatgcccgacgtctggctcaccatgctcagcatgatcgtaggtgccacatgctacgccatgttcatcggccatgccacggcactcatccagtccctggactcttcccggcgtcagtaccaggagaagtacaagcaggtggagcagtacatgtccttccacaagctgccagcagacacgcggcagcgcatccacgagtactatgagcaccgctaccagggcaagatgttcgatgaggaaagcatcctgggcgagctgagcgagccgcttcgcgaggagatcattaacttcacctgtcggggcctggtggcccacatgccgctgtttgcccatgccgaccccagcttcgtcactgcagttctcaccaagctgcgctttgaggtcttccagccgggggatctcgtggtgcgtgagggctccgtggggaggaagatgtacttcatccagcatgggctgctcagtgtgctggcccgcggcgcccgggacacacgcctcaccgatggatcctactttggggagatctgcctgctaactaggggccggcgcacagccagtgttcgggctgacacctactgccgcctttactcactcagcgtggaccatttcaatgctgtgcttgaggagttccccatgatgcgccgggcctttgagactgtggccatggatcggctgctccgcatcggcaagaagaattccatactgcagcggaagcgctccgagccaagtccaggcagcagtggtggcatcatggagcagcacttggtgcaacatgacagagacatggctcggggtgttcggggtcgggccccgagcacaggagctcagcttagtggaaagccagtactgtgggagccactggtacatgcgccccttcaggcagctgctgtgacctccaatgtggccattgccctgactcatcagcggggccctctgcccctctcccctgactctccagccaccctccttgctcgctctgcttggcgctcagcaggctctccagcttccccgctggtgcccgtccgagctggcccatgggcatccacctcccgcctgcccgccccacctgcccgaaccctgcacgccagcctatcccgggcagggcgctcccaggtctccctgctgggtccccctccaggaggaggtggacggcggctaggacctcggggccgcccactctcagcctcccaaccctctctgcctcagcgggcaacaggcgatggctctcctgggcgtaagggatcaggaagtgagcggctgcctccctcagggctcctggccaaacctccaaggacagcccagccccccaggccaccagtgcctgagccagccacaccccggggtctccagctttctgccaacatgtaaaacctttgagtacatccagccttagttcttggggtgcagtagtatgtacccaagggcagatgcctcttggggaaggccatggggacctgaaacattgccccatggaaatgtcgaccctgtgcggacattccgcatactgccatgaagacggtctctgtgtcctcagctcaagaatcctgtagcttgtcccatcataatccattcacccgttcatcatgtgtactgagcagctaccatgttcaaggtaatatgccaggcgctgtatgtctccactgccaagtagaagtgactcaaaaccctctgacaaggatattcccttggctatggtcctgccaggtgcaggcccaggcccatgaccccacctttactaagcacaagtacttgccactgccatcactgccaagtaactagatgtctctgtttccctgccaatgatcctgcaggttctgcccggtctggttatcttcctgttcctgtagcatagccaggcactgccagtcacctgtgcccccattgctgtcagcagatgtcttgggtcctgagtgtgggtatccacttttacccgctcactgccacctgtggacactctgtgtctaccctctgagtgggaacatacttctaagttccctgcagtctctgtcctgtggtagaccatctttttgtaaactgcgagcttcctcttccctgtaccctctgccccagtcgtgaccccctaaaagttaaggggtagttggcacctccttattaatatgccagcctagatcccccccggtggaggggcaaatggctgaatccttgtgtgatatttttttcttcgcttgtttatttattcatttatttaattgtatttattcatttactaactttatgtgttaccaattaattttgtttacccattcctttatccatccctcccctccttttcaggtaaggagacaggaggagtaggaggaggcagggcctctccatgccagcctctgtggtccttgcccaaacccatcagcgcaatacttgaaccttctcccaggtaggggcaggaggagccacatgagagagggagaaggaccgcgtttacctttagagttttgttttgttttttccttctgagtttgctgttggtgcaggaataagggaaaggcccaaggtatccaagcctggggaagggcaggccagccagcacctctgccttctcagggacaagagtagtcctttaccaccctcactctgcctgtcccctctcctactctacagcattaaagactgtgggaccaggaccctaagtctcctttccttctgggtggggagttctggggttcttggtgtgtgggagaagttttataattgcttccaaacagctgggtttaaatataaaatagacacactc

SEQ ID NO: 8 - protein, 703 aa, Homo sapiens
EIEQERVKSAGAWIIHPYSDFRFYWDLIMLLLMVGNLIVLPVGITFFKEENSPPWIVFNVLSDTFFLLDLVLNFRTGIVVEEGAEILLAPRAIRTRYLRTWFLVDLISSIPVDYIFLVVELEPRLDAEVYKTARALRIVRFTKILSLLRLLRLSRLIRYIHQWEEIFHMTYDLASAVVRIFNLIGMMLLLCHWDGCLQFLVPMLQDFPPDCWVSINHMVNHSWGRQYSHALFKAMSHMLCIGYGQQAPVGMPDVWLTMLSMIVGATCYAMFIGHATALIQSLDSSRRQYQEKYKQVEQYMSFHKLPADTRQRIHEYYEHRYQGKMFDEESILGELSEPLREEIINFTCRGLVAHMPLFAHADPSFVTAVLTKLRFEVFQPGDLVVREGSVGRKMYFIQHGLLSVLARGARDTRLTDGSYFGEICLLTRGRRTASVRADTYCRLYSLSVDHFNAVLEEFPMMRRAFETVAMDRLLRIGKKNSILQRKRSEPSPGSSGGIMEQHLVQHDRDMARGVRGRAPSTGAQLSGKPVLWEPLVHAPLQAAAVTSNVAIALTHQRGPLPLSPDSPATLLARSAWRSAGSPASPLVPVRAGPWASTSRLPAPPARTLHASLSRAGRSQVSLLGPPPGGGGRRLGPRGRPLSASQPSLPQRATGDGSPGRKGSGSERLPPSGLLAKPPRTAQPPRPPVPEPATPRGLQLSANM

SEQ ID NO: 9 - DNA, 18 bp, Homo sapiens
ggccatgctgaccaagct

SEQ ID NO: 10 - DNA, 18 bp, Homo sapiens
gtgccttcgcggatgatg

SEQ ID NO: 11 - DNA, 22 bp, Homo sapiens
tcacccggctggaagacctcga

SEQ ID NO: 12 - DNA, 23 bp, Homo sapiens
tgagacagcagatagagccaagc

SEQ ID NO: 13 - DNA, 22 bp, Homo sapiens
tccctgccaatttgacatcttc

SEQ ID NO: 14 - DNA, 26 bp, Homo sapiens
catcaccattggcaatgagcggttcc

SEQ ID NO: 15 - DNA, 24 bp, Artificial Sequence
  RDA primer
agcactctccagcctctcaccgca

SEQ ID NO: 16 - DNA, 12 bp, Artificial Sequence
  RDA primer
gatctgcggtga

SEQ ID NO: 17 - DNA, 24 bp, Artificial Sequence
  RDA primer
accgacgtcgactatccatgaaca

SEQ ID NO: 18 - DNA, 12 bp, Artificial Sequence
  RDA primer
gatctgttcatg

SEQ ID NO: 19 - DNA, 24 bp, Artificial Sequence
  RDA primer
aggcaactgtgctatccgagggaa

SEQ ID NO: 20 - DNA, 12 bp, Artificial Sequence
  RDA primer
gatcttccctcg

SEQ ID NO: 21 - DNA, 54 bp, Artificial Sequence
  SSH adapter oligonucleotide
ctaatacgactcactatagggctcgagcggccgcccgggcaggtacctgcccgg

SEQ ID NO: 22 - DNA, 52 bp, Artificial Sequence
  SSH adapter oligonucleotide
ctaatacgactcactatagggcagcgtggtcgcggccgaggtacctcggccg

SEQ ID NO: 23 - DNA, 22 bp, Artificial Sequence
  PCR primer
ctaatacgactcactatagggc

SEQ ID NO: 24 - DNA, 22 bp, Artificial Sequence
  PCR nested primer
tcgagcggccgcccgggcaggt

SEQ ID NO: 25 - DNA, 22 bp, Artificial Sequence
  PCR primer
ctaatacgactcactatagggc

SEQ ID NO: 26 - DNA, 20 bp, Artificial Sequence
  PCR nested primer
agcgtggtcgcggccgaggt